(19) United States
(12) Patent Application Publication
(10) Pub. No.: US 2026/0232864 A1
(43) Pub. Date: Aug. 13, 2026

(54) DEVICE AND METHOD FOR TREATING SKIN INJURIES

(71) Applicant: Advanced Wound Systems, Inc., Delray Beach, FL (US)

(72) Inventors: Andrew M. Klapper, Boynton Beach, FL (US); Michael S. Risin, Palm Beach, FL (US)

(73) Assignee: Advanced Wound Systems, Inc., Delray Beach, FL (US)

(21) Appl. No.: 19/156,946

(22) PCT Filed: Feb. 21, 2024
(86) PCT No.: PCT/US2024/016640

§ 371 Date: Aug. 15, 2025

Related U.S. Application Data
(60) Provisional application No. 63/486,124, filed on Feb. 21, 2023.

(51) Int. Cl. A61L 15/64; A61F 13/02; A61F 13/0246; A61L 15/22; A61L 15/42; A61L 15/44; A61L 15/58

(52) U.S. Cl. CPC A61L 15/64; A61F 13/0243; A61F 13/0246; A61L 15/225; A61L 15/425; A61L 15/44; A61L 15/58; A61L 2300/404

(57) ABSTRACT

Provided are devices and methods for treating skin injuries. The devices comprise: (a) an adhesive material and a mesh scaffold configured to structurally support a skin injury of a subject, wherein the adhesive material is configured to affix the mesh scaffold directly over the skin injury of the subject. The devices are configured to provide primary closure or delayed primary closure of the skin injury. The method comprises a use of the device.

### CROSS REFERENCE

This application claims the benefit of U.S. Provisional Application No. 63/486,124, filed on Feb. 21, 2023, which is incorporated herein by reference in its entirety.

### BACKGROUND

Skin tears are commonly treated by reapproximation with fixation using a plurality of single-planar wound strips (e.g., Steri Strips) at the wound edges, then covering the wound strips and skin tear with an overlying bandage. The observed failure rate for this method of treatment is high. Failure to treat the skin tear results in obligatory surgical debridement of devitalized tissues and a prolonged and expensive course of secondary healing. Moreover, subjects at increased risk for traumatic skin injuries or skin tears include the elderly, critically ill/injured, or malnourished. Such subjects often have fragile skin, which may be too fragile for staples, sutures or wound strips to successfully treat a skin injury.

### SUMMARY

Aspects of the disclosure herein provide a skin wound dressing comprising a mesh scaffold having a top side and a bottom side, and an adhesive layer associated with the bottom side of the scaffold, wherein at least one of the mesh scaffold and adhesive layer comprise a plurality of fenestrations configured to allow for fluid egress when the dressing is placed over a skin injury of a subject. In some embodiments, the mesh scaffold comprises a non-absorbable mesh. In some embodiments, the mesh scaffold comprises an absorbable mesh. In some embodiments, the mesh scaffold comprises at least one of a polyester-based, polypropylene-based, silk-based, nylon-based, spandex-based, polyglactin-based and polyglycolic acid (PGA)-based mesh.

Provided herein is a skin wound dressing comprising a polyester-based or polypropylene-based mesh scaffold having a top side and a bottom side, and an adhesive layer associated with the bottom side of the scaffold, wherein the dressing has a porosity configured to allow for fluid egress when the dressing is placed over a skin injury of a subject. In some embodiments, at least one of the mesh scaffold and the adhesive layer comprises a plurality of fenestrations.

In some embodiments, the mesh scaffold has a low absorbance or no absorbance of wound exudate from the skin injury. In some embodiments, the mesh scaffold absorbs less than 50% of wound exudate from the skin injury. In some embodiments, the mesh scaffold has a low biodegradability when the dressing is placed over the skin injury. In some embodiments, the mesh scaffold maintains at least 50% of one or more mechanical properties after 1, 2, 3, 4, 5, 6, or 7 days of covering the skin injury as compared to the mesh scaffold before covering the skin injury. In some embodiments, the one or more mechanical properties comprises tensile modulus, tensile strength, tensile strain, elastic modulus, stiffness, or any combination thereof. In some embodiments, the porosity is about 0.001 mm to about 20 mm, about 0.01 mm to about 10 mm, about 0.05 mm to about 5 mm, or about 0.1 mm to about 5 mm. In some embodiments, fenestration of the plurality of fenestrations has a linear shape, a rectangular shape, a circular shape, an elliptical shape, a triangular shape, a trapezoidal shape, or a cross shape. In some embodiments, the plurality of fenestrations is arranged in a regular pattern having an offset distance between a first fenestration and a second fenestration. In some embodiments, the plurality of fenestrations is arranged in an irregular pattern. In some embodiments, the plurality of fenestrations is configured to allow for fluid egress when the dressing is placed over the skin injury. In some embodiments, the adhesive layer is configured to adhere the device to the subject for at least 1, 2, 3, 4, 5, 6, or 7 days. In some embodiments, the skin injury comprises a skin flap or a skin graft. In some embodiments, the dressing provides multiplanar or multiaxial immobilization of the skin flap or the skin graft to the skin injury. In some embodiments, a surface area of the skin injury is at least 1, 2, 3, 4, 5, 6, 7, 8, 9, 10, 15, or 20 cm². In some embodiments, a surface area of the skin flap or the skin graft is at least 1, 2, 3, 4, 5, 6, 7, 8, 9, 10, 15, or 20 cm². In some embodiments, the dressing is configured to adhere across the surface area of the skin injury continuously for at least 1, 2, 3, 4, 5, 6, or 7 days. In some embodiments, the adhesive layer comprises an adhesive tape, film, glue, spray, gel, foam, or any combination thereof applied to the bottom side of the mesh scaffold. In some embodiments, the mesh scaffold has a pore size between about 0.01 mm to about 10 mm, about 0.05 mm to about 5.0 mm, about 0.8 mm to about 4.0 mm, or about 1.0 mm to about 2.0 mm. In some embodiments, the mesh scaffold has a weight of between about 5 grams per square meter (GSM) to about 200 GSM. In some embodiments, the mesh scaffold comprises a thickness of between about 0.01 mm to about 10 mm, about 0.05 mm to about 5 mm, or about 0.1 mm to about 1 mm. In some embodiments, the plurality of fenestrations is at least one of punched, cut, stamped, punctured, formed, sprayed, seared, burned, melted, and lasered through the mesh scaffold, the adhesive layer, a third layer, or any combination thereof. In some embodiments, the fenestration pattern is configured to preserve immobilization capacity of the dressing. In some embodiments, the skin wound dressing further comprises a non-adhesive backing layer attached to the adhesive layer. In some embodiments, the skin wound dressing further comprises a non-adherent overlay layer attached to the top side of the scaffold and configured to prevent at least one of a bandage, a compression bandage, an absorbent covering, a drape, a textile, and an article of clothing contacting the skin wound dressing from at least partially adhering to the dressing. In some embodiments, the dressing comprises an antimicrobial, a silver antimicrobial, a silver material, an antimicrobial gel foam, an active ingredient, an antibiotic, an antifungal, a wound care product, a biologic, a medicament, an antiseptic, an anesthetic, an analgesic, a therapeutic, a hemostatic agent, or any combination thereof. In some embodiments, the mesh scaffold is woven or knitted.

Provided herein is a method of treating a skin injury comprising a wound bed, the method comprising covering a skin flap of the skin injury or a skin graft applied to the wound bed of the skin injury with a piece of a skin wound dressing, wherein the skin wound dressing comprises a mesh scaffold having a top side and a bottom side; and an adhesive layer associated with the bottom side of the scaffold, wherein the skin wound dressing has a porosity configured to allow for fluid egress when the skin wound dressing is placed over a skin injury of a subject; and providing multiplanar or multiaxial immobilization of the skin flap or the skin graft with the skin wound dressing. In some embodiments, at least one of the mesh scaffold and the adhesive layer of the skin wound dressing comprises a plurality of fenestrations.

Provided herein is a method of treating a skin injury of a subject comprising adhering a piece of a skin wound dressing directly over the skin injury, the skin wound dressing comprising a mesh scaffold having a top side and a bottom side; and an adhesive layer associated with the bottom side of the scaffold, wherein the at least one of the mesh scaffold and adhesive layer comprises a plurality of fenestrations configured to allow for fluid egress when the skin wound dressing is placed over the skin injury of the subject; and providing multiplanar or multiaxial immobilization of the skin injury with the skin wound dressing.

In some embodiments, the method comprises treating a skin injury wherein the skin injury comprises a skin flap or a skin graft atop a wound bed of the skin injury. In some embodiments, the mesh scaffold comprises a non-absorbable mesh. In some embodiments, the mesh scaffold comprises an absorbable mesh. In some embodiments, the mesh scaffold comprises at least one of a polyester-based, polypropylene-based, silk-based, nylon-based, spandex-based, polyglactin-based and polyglycolic acid (PGA)-based mesh. In some embodiments, the mesh scaffold absorbs less than 50% of wound exudate from the skin injury. In some embodiments, the mesh scaffold has a low biodegradability when the skin wound dressing is placed over the skin injury. In some embodiments, the mesh scaffold maintains at least 50% of one or more mechanical properties after 1, 2, 3, 4, 5, 6, or 7 days of covering the skin injury as compared to the mesh scaffold before covering the skin injury. In some embodiments, the one or more mechanical properties comprises tensile modulus, tensile strength, tensile strain, elastic modulus, stiffness, or any combination thereof. In some embodiments, the method comprises providing multiplanar or multiaxial immobilization of the skin flap or the skin graft with the skin wound dressing until the skin flap or the skin graft is at least partially attached to the wound bed. In some embodiments, the method comprises preventing disruption of wound repair due to shear forces, blistering, subdermal fluid accumulation, hematoma, sub graft fluid accumulation, or any combination thereof with the skin wound dressing. In some embodiments, the method comprises covering at least about 50% to about 100% of the surface area of the skin flap or the skin graft with the piece of the skin wound dressing. In some embodiments, the method comprises adhering the skin wound dressing across the skin injury continuously for at least 1, 2, 3, 4, 5, 6, or 7 days. In some embodiments, the method comprises removing the skin wound dressing from the skin injury once the skin flap or skin graft is at least partially attached to the wound bed of the subject. In some embodiments, the method comprises removing the skin wound dressing from the skin injury once the skin injury exhibits a marker of healing. In some embodiments, the marker of healing comprises a cessation of wound drainage from the skin injury, a cell layer of closure at an injured edge of the skin injury, an indicator of skin viability, blanching of the skin injury with pressure, pink coloration of the skin injury, or any combination thereof.

Provided herein is a method of making a skin wound dressing, the method comprising applying an adhesive layer to a bottom side of a polyester-based or polypropylene-based mesh scaffold, wherein the skin wound dressing has a porosity configured to allow for fluid egress when the skin wound dressing is placed over the skin injury of a subject. In some embodiments, the method comprises adding a plurality of fenestrations to the mesh scaffold or adhesive layer. In some embodiments, the method comprises applying an adhesive layer to a bottom side of a mesh scaffold and adding a plurality of fenestrations to the mesh scaffold or adhesive layer configured to allow for fluid egress when the skin wound dressing is placed over the skin injury of a subject. In some embodiments, the fenestration pattern is configured to preserve immobilization capacity of the skin wound dressing. In some embodiments, the adhesive layer comprises an adhesive tape, film, glue, spray, gel, foam, or any combination thereof. In some embodiments, the method comprises affixing a backing layer to the adhesive layer. In some embodiments, the method comprises applying a non-adhesive overlay to a top side of the mesh scaffold.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 depicts an exemplary device of the present disclosure and a method for coupling a scaffold to an adhesive material.

FIGS. 2A-2C illustrates treatment of an exemplary skin injury of a subject using the devices and methods described herein. FIG. 2A depicts a skin tear with a skin flap. FIG. 2B depicts an exemplary device of the present disclosure affixed directly over the skin tear and the skin flap. FIG. 2C depicts the healed skin injury.

FIGS. 3A-3B depict a non-adhesive backing layer of the device configured to removably adhere to an exemplary device of the present disclosure. FIG. 3A depicts a device lacking a non-adhesive backing layer. FIG. 3B depicts a non-adhesive backing layer configured to removably adhere to a device.

FIGS. 4A-4B depict a device comprising two pieces of a non-adhesive backing layer. FIG. 4A depicts a device without fenestrations. FIG. 4B depicts a device with fenestrations added.

FIGS. 5A-5B depict a device comprising three pieces of a non-adhesive backing layer. FIG. 5A depicts a device without fenestrations. FIG. 5B depicts a device with fenestrations added.

FIGS. 6A-6C depict exemplary scaffolds of the present disclosure. FIG. 6A depicts an exemplary mesh. FIG. 6B depicts a scaffold comprising a plurality of circular pores. FIG. 6C depicts a scaffold comprising a plurality of square-shaped pores.

FIGS. 7A-7B depict exemplary trilaminate devices of the present disclosure. FIG. 7A depicts an exemplary trilaminate device comprising a plurality of pores and three pieces of a non-adhesive backing layer. FIG. 7B depicts an exemplary trilaminate device comprising a plurality of fenestrations and two pieces of a non-adhesive backing layer.

FIGS. 8A-8B depict perspective exploded and elevational views, respectively, of an exemplary non-occlusive trilaminate device of the present disclosure comprising a free edge. FIG. 8A depicts a perspective exploded view of each layer of a non-occlusive trilaminate device showing a plurality of fenestrations through each layer. FIG. 8B depicts an elevational view of the exemplary non-occlusive trilaminate device showing a free edge and a non-adhesive backing layer comprising two pieces.

FIGS. 9A-9B depict perspective exploded and elevational views, respectively, of an exemplary non-occlusive trilaminate device of the present disclosure comprising a free edge and a non-adhesive backing layer comprising “handle bars” that a user of the device to position the device before affixing the device to the subject. FIG. 9A depicts a perspective exploded view of each layer of a non-occlusive trilaminate device showing a plurality of fenestrations through each layer. FIG. 9B depicts an elevational view of the exemplary non-occlusive trilaminate device showing a free edge and a non-adhesive backing layer comprising three pieces.

FIGS. 10A-10C depict a skin injury comprising a skin tear treated using a device and method of the present disclosure. FIG. 10A depicts a skin tear comprising a skin flap. FIG. 10B depicts an exemplary device affixed directly over a skin flap. FIG. 10C depicts the healed skin tear after 2 weeks of treatment using a device and method of the present disclosure.

FIGS. 11A-11C depict a minor skin injury comprising a laceration treated using a device and method of the present disclosure. FIG. 11A depicts a skin laceration of a subject. FIG. 11B depicts an exemplary device affixed directly over the skin laceration. FIG. 11C depicts the healed skin laceration after 1 week of treatment using a device and method of the present disclosure.

FIGS. 12A-12C depict a major skin injury comprising a full thickness skin tear treated using a device and method of the present disclosure. FIG. 12A depicts a skin tear comprising a partial skin flap. FIG. 12B depicts an exemplary device affixed directly over the skin tear. FIG. 12C depicts complete survival of the injured skin after 2 weeks of treatment using a device and method of the present disclosure.

FIGS. 13A-13C depict a skin tear comprising a skin flap treated using a device and method of the present disclosure. FIG. 13A depicts a skin tear comprising a skin flap having an injured edge. FIG. 13B depicts an exemplary device affixed directly over a skin flap. FIG. 13C depicts the healed skin tear following seven days of treatment using a device and method of the present disclosure.

FIGS. 14A-14D depict treatment of a skin tear and hematoma using a non-occlusive device and a method of the present disclosure. FIG. 14A depicts the skin tear comprising a partial skin flap. FIG. 14B depicts an exemplary device affixed directly over the skin tear and underlying hematoma. FIG. 14C depicts complete survival of the partial skin flap tissue after seven days and following removal of the non-occlusive device. FIG. 14D depicts the progress of wound healing two weeks after removal of the non-occlusive device.

FIGS. 15A-15D depict treatment of a skin injury comprising two partial skin flaps using a non-occlusive device and a method of the present disclosure. FIG. 15A depicts the skin injury comprising two partial skin flaps. FIG. 15B depicts an exemplary device affixed directly over the two skin tears after they are joined together by several sutures. FIG. 15C depicts complete survival of tissue from both partial skin flaps after seven days and following removal of the non-occlusive device. FIG. 15D depicts the progress of wound healing two weeks after removal of the non-occlusive device.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term ‘about’, in reference to a number, refers to the number plus or minus 10% of the number. The term ‘about’, in reference to a range, refers to the range minus 10% of its lowest value and plus 10% of its greatest value.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of obtaining a measurement and include determining if an element is present or not (for example, detection). These terms can include quantitative, qualitative, or quantitative and qualitative determinations. Assessing can be relative or absolute. “Detecting the presence of,” as used herein, includes determining the amount of something present, as well as determining whether it is present or absent.

The terms “subject,” “individual,” or “patient” are often used interchangeably herein. A subject, individual, or patient can be a biological entity containing expressed genetic materials. In some embodiments, the subject, individual, or patient can be an animal, mammal, or human.

As used herein, the terms “treatment” or “treating” are used in reference to an intervention regimen for obtaining beneficial or desired results in the recipient. Beneficial or desired results include but are not limited to a therapeutic benefit and/or a prophylactic benefit. In some embodiments, a therapeutic benefit refers to prevention or amelioration of symptoms or of an underlying disorder being treated. Also, a therapeutic benefit can be achieved with prevention or amelioration of one or more of the physiological symptoms associated with the underlying disorder such that an improvement is observed in the subject, notwithstanding that the subject could still be afflicted with the underlying disorder. A prophylactic effect includes delaying, preventing, or eliminating the appearance of a disease or a condition, delaying or eliminating the onset of symptoms of a disease or a condition, slowing, halting, or reversing the progression of a disease or a condition, or any combination thereof. For prophylactic benefit, a subject at risk of developing a particular disease or a condition or reporting one or more of the physiological symptoms of a disease or a condition can undergo treatment.

### Skin Injuries

Provided herein are devices and methods for treating a skin injury of a subject. In some embodiments, the devices and methods described herein provide primary closure of a skin injury. As used in the specification and claims, the term “primary closure” refers to direct repair or closure of a skin injury or direct anchoring of a graft to a skin injury. In some embodiments, the skin injury comprises a skin tear, a skin flap, an avulsed skin flap, a skin laceration, a skin blister, a skin graft, a tissue graft, a biocompatible material graft, a skin abrasion, a skin incision, a burn, a surgical wound or incision, or any combination thereof. In some embodiments, the skin injury comprises a mucosal injury (e.g., a skin flap of the gum associated with removal of a wisdom tooth). In some embodiments, the skin injury comprises an acute wound, a subacute wound, or a chronic wound. In some embodiments, the skin injury comprises a traumatic skin injury.

In some embodiments, the devices and methods described herein provide secondary closure of a skin injury.

In some embodiments, the devices and methods described herein provide delayed primary closure. In some embodiments, the skin injury can be cleaned, debrided, and antimicrobials are applied to the skin injury before the device is applied directly over the skin injury. In some embodiments, the skin injury can be observed for a period of time to ensure no infection is apparent before the skin injury is closed using the devices and methods provided herein.

Traumatic skin injuries frequently include a skin tear. A skin tear is a type of skin injury caused by shear, friction, or blunt forces that results in a separation of skin layers. The skin tear may be a partial thickness wound (e.g., the epidermis is separated from the dermis) or a full thickness wound (e.g., the epidermis and the dermis are separated from the underlying structures, including fat, muscle, or fascia). Overall, skin tears are highly variable in presentation between subjects based on their depth, skin flap status, associated tissue necrosis, infection, underlying hematoma, or overall amount of viable tissue surrounding the wound.

Some skin tears retain a skin flap. The skin flap can be composed of detached layers of epidermis or dermis. To treat a skin tear, any existing skin flap can be reattached to the wound bed of the subject. For some skin tears, the edges of the skin flap can be realigned to a normal anatomical position. For some skin tears, one or more edges of the skin flap cannot be realigned to the normal anatomical position. The tissue of a skin flap that is most distant from the blood supply is at the highest risk of necrosis or failing to attach. Since skin flaps often do not have reliable blood supplies, it is provided herein that skin tears should be subject to the basic tenets of skin grafting to improve their survival.

Provided herein are devices and methods for treating a skin injury of a subject. In some embodiments, the skin injury comprises a skin tear. In some embodiments, the devices and methods described herein provide primary closure or delayed primary closure of a skin injury, wherein the skin injury comprises a skin tear. In some embodiments, the skin tear comprises a partial thickness wound. In some embodiments, the skin tear comprises a full thickness wound. In some embodiments, the skin injury comprises a separation or uncoupling of a first skin layer from a second skin layer. In some embodiments, the skin injury comprises a separation or uncoupling of one or more skin layers from an underlying skin layer (e.g., the cutis from the subcutis). In some embodiments, the skin injury comprises a separation or uncoupling of the epidermis of the subject from the dermis of the subject. In some embodiments, the skin injury comprises a separation or uncoupling of the epidermis and the dermis of the subject from an underlying bodily tissue of the subject. In some embodiments, the underlying bodily tissue of the subject comprises at least one of fat, muscle, or fascia of the subject underlying or adjacent to the skin injury.

In some embodiments, skin tears do not have a skin flap (e.g., a skin flap can be completely torn off the subject). For severe or traumatic wounds, treating a skin injury may require providing a graft (e.g., skin or tissue) atop exposed tissue layers (e.g., the exposed subcutis or fascia) of the wound bed. In some embodiments, the graft can be required to supplement or replace an absent or minimal skin flap of a skin tear. In some embodiments, a graft can be required underneath a skin flap to promote or improve wound healing of the skin injury.

Provided herein are devices and methods for treating a skin injury of a subject. In some embodiments, the method comprises adding a graft to the skin injury (e.g., a skin graft or a tissue graft). In some embodiments, the devices and methods described herein provide primary closure of a graft treating a skin injury. In some embodiments, the graft comprises a biocompatible graft, a skin graft, a tissue graft, an amniotic graft, a composite graft, a cell graft, a biocompatible material graft, a synthetic skin graft, a synthetic tissue graft, an exoskeletal support piece, or any combination thereof. In some embodiments, the graft comprises a split-thickness graft, a full-thickness graft, a composite graft, or any combination thereof. In some embodiments, the graft can be applied to a wound bed of the subject. In some embodiments, the graft supplements or replaces a partial or absent skin flap of a skin tear. In some embodiments, the skin injury of the subject comprises the wound bed. In some embodiments, a graft can be provided to the wound bed underneath a skin flap. In some embodiments, the method comprises applying a graft to the skin injury, the device, or any combination thereof before affixing the device directly over the skin injury of the subject.

Wound repair of a traumatic skin injury frequently includes reattachment of a skin flap to a wound bed of the subject or attachment of a graft to the wound bed of the subject. Wound repair can be disrupted by shear, friction, or blunt forces against or adjacent to the skin. Wound repair could also be disrupted by blistering, subdermal fluid accumulation, hematoma, or sub graft fluid accumulation at or near the skin injury of the subject. The device disclosed herein can promote wound repair, epithelialization at an injured edge of a skin injury, and effective healing of a skin injury by immobilizing or structurally supporting the skin injury (e.g., by providing multiaxial or multiplanar immobilization) and directly repairing the skin injury or anchoring a graft to the skin injury. In some embodiments, the device can distribute a pressure or force equal to or greater than the force necessary to tear the skin injury over a larger area. In some embodiments, the device, the adhesive material, the scaffold, or any combination thereof can provide a tensile force greater than that required to cause skin injury or disrupt wound repair of a skin injury. In some embodiments, structurally supporting comprises anchoring the skin flap to the wound bed. In some embodiments, immobilizing comprises providing adhesion and tension.

### Devices and Methods

### Devices

Provided herein is a non-occlusive device comprising a mesh scaffold having a top side and a bottom side, and an adhesive layer associated with the bottom side of the scaffold, wherein the non-occlusive device has a porosity configured to allow for fluid egress when the dressing is placed over a skin injury of a subject.

As used herein, a “non-occlusive” device can mean a device (e.g., skin wound dressing) having a porosity configured to allow for fluid egress when the dressing is placed over a skin injury of a subject. In some embodiments, the non-occlusive device comprises a plurality of fenestrations through at least one of the scaffold and adhesive layer. The plurality of fenestrations can be configured to allow for fluid egress when the skin wound dressing is placed over the skin injury.

Fluid draining from a healing skin injury (e.g., wound exudate) can disrupt the healing process if the fluid cannot adequately drain away from the skin injury (e.g., drain from the wound bed) or is prevented from draining (e.g., by an occlusive covering over the skin injury). Provided herein is a non-occlusive device configured to allow for drainage of fluid (e.g., wound exudate) from a skin injury, wherein fluid passes through the device without the need to remove or reapply the device over the skin injury of the subject. The non-occlusive device is configured to cover the skin injury continuously during the healing process. In some embodiments, the skin wound dressing provided herein can cover the skin injury continuously for at least 1, 2, 3, 4, 5, 6, or 7 days; until the wound exhibits a marker of healing; or until a skin flap or skin graft of the skin injury at least partially attaches to an underlying wound bed.

In some embodiments, the porosity of the non-occlusive device is about 0.001 mm to about 20 mm, about 0.01 mm to about 10 mm, about 0.05 mm to about 5 mm, or about 0.1 mm to about 5 mm. In some embodiments, the porosity is at least about 0.001 mm, at least about 0.01 mm, at least about 0.05 mm, at least about 0.1 mm, at least about 0.5 mm, at least about 1 mm, at least about 2 mm, at least about 3 mm, at least about 4 mm, at least about 5 mm, at least about 10 mm, at least about 15 mm, or at least about 20 mm. A larger porosity increases fluid egress when the skin wound dressing is placed over the skin injury. In some cases, adding a plurality of fenestrations to at least one of the mesh scaffold and adhesive layer of the skin wound dressing can increase the porosity of the skin wound dressing.

In some cases, a mesh scaffold of the skin wound dressing has a pore size, pore shape, pore pattern, or any combination thereof configured to allow for fluid egress when the dressing is placed over a skin injury of a subject. In some embodiments, the mesh scaffold has a pore size between about 0.01 mm to about 10 mm, about 0.05 mm to about 5.0 mm, about 0.8 mm to about 4.0 mm, or about 1.0 mm to about 2.0 mm. In some embodiments, the pore size is at least about 0.01 mm, 0.05 mm, 0.10 mm, 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, or 0.95 mm. In some embodiments, the pore size is at least about 1.0 mm, 1.2 mm, 1.4 mm, 1.6 mm, 1.8 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5.0 mm. In some embodiments, the pore size is at least about 6 mm, 7 mm, 8 mm, 9 mm, or 10 mm.

In some embodiments, the mesh scaffold of the skin wound dressing has a density (e.g., weight) or thickness configured to allow for fluid egress when the skin wound dressing is placed over a skin injury of a subject. In some embodiments, the mesh scaffold has a weight of between about 5 grams per square meter (GSM) to about 200 GSM. In some embodiments, the mesh scaffold has a weight of about 30 GSM to about 70 GSM. In some embodiments, the mesh scaffold has a weight of at most about 5, 6, 7, 8, 9, 10, 15, 20, 25, 30, 35, 40, 45, 50, 55, 60, 65, 70, 75, 80, 85, 90, 95, or 100 GSM. In some embodiments, the mesh scaffold has a weight of at most about 110, 120, 130, 140, 150, 160, 170, 180, 190, or 200 GSM. In some embodiments, the mesh scaffold has a thickness of between about 0.01 mm to about 10 mm, about 0.05 mm to about 5 mm, or about 0.1 mm to about 1 mm. In some embodiments, the mesh scaffold has a thickness of at most about 0.01 mm, 0.02 mm, 0.03 mm, 0.04 mm, 0.05 mm, 0.06 mm, 0.07 mm, 0.08 mm, 0.09 mm, 0.10 mm, 0.11 mm, 0.12 mm, 0.13 mm, 0.14 mm, 0.15 mm, 0.16 mm, 0.17 mm, 0.18 mm, or 0.19 mm. In some embodiments, the mesh scaffold has a thickness of at most about 0.20 mm, 0.25 mm, 0.30 mm, 0.35 mm, 0.40 mm, 0.45 mm, 0.50 mm, or 0.55 mm. In some embodiments, the mesh scaffold has a thickness of at most about 0.6 mm, 0.7 mm. 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm.

The scaffold can be durable enough to maintain at least about 50%, 60%, 70%, 80%, or 90% of one or more mechanical properties after at least 1, 2, 3, 4, 5, 6, or 7 days of covering the skin injury as compared to the mesh scaffold before covering the skin injury. In some cases, the scaffold is durable enough to maintain at least 91%, 92%, 93%, 94%, 95%, 96%, 97%, 98%, or 99% of the one or more mechanical properties after at least 1, 2, 3, 4, 5, 6, or 7 days of covering the skin injury as compared to the mesh scaffold before covering the skin injury. In some cases, the scaffold is durable enough to maintain at least 50% to 99% of the one or more mechanical properties after at least about 30 days of covering the skin injury as compared to the mesh scaffold before covering the skin injury. The one or more mechanical properties can be tensile modulus, tensile strength, tensile strain, elastic modulus, stiffness, or any combination thereof.

In some embodiments, the mesh scaffold comprises a polyester-based or polypropylene-based mesh. In some embodiments, the mesh scaffold comprises a polyester, polypropylene, silk, nylon, or spandex fiber or mesh. In some cases, the mesh scaffold has a low absorbance or no absorbance of wound exudate from the skin injury. Low absorbance or no absorbance can prevent the mesh scaffold from quickly degrading after being exposed to wound exudate (e.g., supports maintaining at least one of the one or more mechanical properties). In some embodiments, the mesh scaffold absorbs less than 50% of wound exudate from the skin injury. In some embodiments, the mesh scaffold has a low biodegradability when the dressing is placed over the skin injury.

An absorbable mesh scaffold can be used if the absorbable mesh scaffold maintains at least about 50%, 60%, 70%, 80%, or 90% of one or more mechanical properties for the duration the skin wound dressing covers the skin injury (e.g., after 1, 2, 3, 4, 5, 6, or 7 days) compared to the absorbable mesh scaffold before covering the skin injury. The one or more mechanical properties comprises tensile modulus, tensile strength, tensile strain, elastic modulus, stiffness, or any combination thereof. In some cases, the mesh scaffold can comprise absorbable and non-absorbable fibers or polymers.

In some cases, the mesh scaffold comprises an absorbable mesh. In some embodiments, the mesh scaffold comprises a polyglactin-based mesh (e.g., Vicryl) or polyglycolic acid (PGA)-based mesh. In some embodiments, the mesh comprises a polyglactin, polyglycolic acid, Bio-A, TIGR Martrix, or Phasix fiber or mesh.

The mesh scaffold can be woven or knitted. In some embodiments, the weaving or knitting is weft-faced or warp-faced. In some embodiments, the mesh scaffold has a warp-knit (e.g., tricot) or weft-knit construction. The mesh can have a denier count of between about 5D to about 1200D, about 10D to about 600D, about 20D to about 200 D, or about 30D to about 100D. In some cases, the mesh scaffold comprises a 3D printed structure.

A plurality of fenestrations can be added to at least one of the mesh scaffold or adhesive layer such that the mesh scaffold maintains at least about 50%, 60%, 70%, 80%, or 90% of the one or more mechanical properties compared to the mesh scaffold before adding the plurality of fenestrations. In some cases, the fenestration pattern is configured to preserve immobilization capacity of the skin wound dressing.

In some cases, a fenestration of the plurality of fenestrations has a linear shape, a rectangular shape, a circular shape, an elliptical shape, a triangular shape, a trapezoidal shape, or a cross shape. In some embodiments, the plurality of fenestrations is arranged in a regular pattern having an offset distance between a first fenestration and a second fenestration. In some embodiments, the plurality of fenestrations is arranged in an irregular pattern.

An adhesive material can be applied to a bottom side of the mesh scaffold. Mesh scaffolds having large pore sizes or lower density (e.g., structural features that can increase the porosity of the skin wound dressing) can have less surface area on the bottom side of the mesh scaffold for applying the adhesive layer. Thus, in a method of manufacturing a skin wound dressing, a mesh scaffold can be selected based on an amount of surface area available for applying the adhesive layer of the skin wound dressing. In some embodiments, an adhesive layer is of the skin wound dressing is configured to adhere the mesh scaffold to the subject for at least 1, 2, 3, 4, 5, 6, or 7 days with minimal attachment failure or loss of attachment between the skin wound dressing and the subject (e.g., edge lift around the perimeter of the skin wound dressing, or segments of adhesive scaffold covering the skin injury losing attachment to the skin injury). The adhesive layer or adhesive material can be configured to adhere to the subject without loss of attachment of the mesh scaffold to the subject due to fluid egress from the skin injury. Fluid accumulation at the skin injury can result in the adhesive material losing attachment to the subject. In some cases, the porosity of the skin wound dressing allows the adhesive layer to adhere to the subject continuously for at least 1, 2, 3, 4, 5, 6, or 7 days.

In some cases, no more than about 1% to about 50% of the adhesive layer within 1 cm of the edge of the device has lifted from the subject after 1, 2, 3, 4, 5, 6, or 7 days of adhering to the subject. In some cases, no more than about 10% to about 20% of the adhesive layer within 1 cm of the edge of the device has lifted from the subject after 1, 2, 3, 4, 5, 6, or 7 days of adhering to the subject. In some cases, at least 50% to 99% of the mesh scaffold adhering to the subject after affixing the skin wound dressing over a skin injury of a subject remains adhered to the subject after 1, 2, 3, 4, 5, 6, or 7 days. In some cases, at least 80% to 90% of the mesh scaffold adhering to the subject after affixing the skin wound dressing over a skin injury of a subject remains adhered to the subject after 1, 2, 3, 4, 5, 6, or 7 days.

The skin wound dressing can cover a surface area of the skin injury, wherein the surface area is at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 cm². In some cases, a piece of the skin wound dressing can cover the surface area. In some cases, multiple pieces of the skin wound dressing can be used to cover the surface area (e.g., two overlapping pieces). In some cases, a sheet of the skin wound dressing can cover a skin injury spanning across an abdomen of a human subject (e.g., 400-500 cm²).

Provided herein are devices, comprising: a) a first layer comprising an adhesive material; and b) a second layer comprising a scaffold configured to structurally support a skin injury of a subject, wherein the first layer is configured to affix the second layer directly over the skin injury of the subject. Also provided herein are devices, comprising: a) an adhesive material; and b) a scaffold configured to structurally support a skin injury of a subject, wherein the adhesive material is configured to affix the scaffold directly over the skin injury of the subject.

The devices of the present disclosure can be manufactured as long rolls (e.g., at least about 20 inches to about 100 inches wide by about 50 yards to about 250 yards long) comprising an adhesive material (e.g., first layer) and a scaffold (e.g., second layer). Pieces can be cut from the rolls to sizes (e.g., skin injury width or length) appropriate for treating a skin injury. In some embodiments, one or more pieces of the device can be affixed directly over a skin injury of a subject. Relatively large pieces of the device can sometimes be referred to herein as “sheets.” In some embodiments, one or more sheets of the device can be affixed directly over a skin injury of a subject.

In some embodiments, the device or the scaffold is self-adhering to the skin injury of the subject. In some embodiments, the device or the scaffold is configured to removably attach to the subject. In some embodiments, the adhesive material can be applied to the device or the scaffold before the device or the scaffold is affixed directly over the skin injury of the subject. In some embodiments, the adhesive material can be applied to the subject before the device or the scaffold is affixed directly over the skin injury of the subject. In some embodiments, an additional adhesive material can be applied to the subject, the skin injury, or the device before the device or the scaffold is affixed directly over the skin injury.

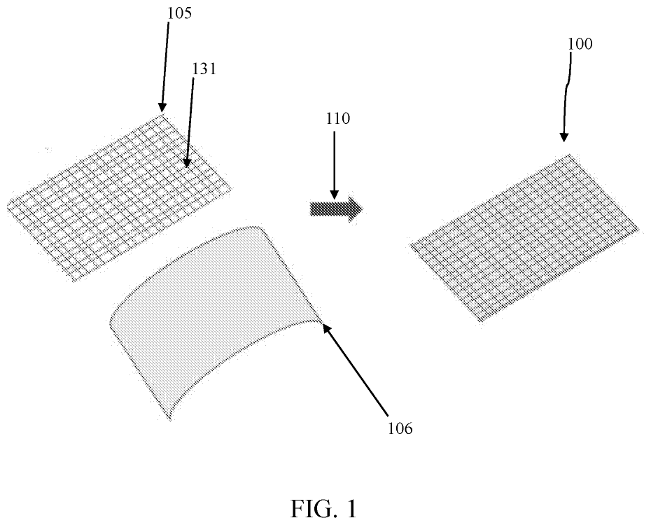

FIG. 1 depicts an exemplary representation of a device 100 of the present disclosure. FIG. 1 depicts a first layer 106 comprising an adhesive material being coupled to a second layer 105 comprising a scaffold by a method 110. In some embodiments, the second layer 105 comprises a plurality of pores 131. In some embodiments, the scaffold can be porous. In some embodiments, the scaffold permits egress of any subdermal fluid through the device and prevents subdermal fluid accumulation.

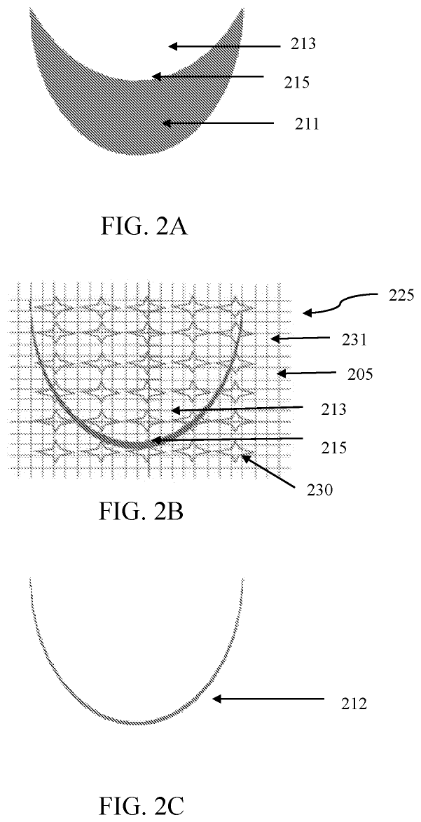

FIG. 2A illustrates an exemplary skin injury of a subject comprising a skin tear 211. In some embodiments, the skin tear 211 comprises a skin flap 213, wherein the skin flap comprises an injured edge 215. FIG. 2B shows an exemplary representation of the device 225 affixed directly over the skin injury. In some embodiments, the device 225 pulls and holds the injured edge 215 of the skin flap 213 into alignment with normal anatomical position. In some embodiments, the device 225 comprises a scaffold (e.g. mesh) 205 affixed directly over the skin flap 213. While the device 225 is affixed directly over the skin tear, the skin flap 213 can be supported by the immobilization properties of the device. In some embodiments, the device 225 prevents subdermal fluid accumulation via permitting egress of any subdermal fluid through a plurality of pores 231 traversing the device 225. In some embodiments, subdermal fluid accumulation under the flap does not disrupt wound repair. In some embodiments, shear, friction, or blunt forces are prevented from disrupting repair of the skin tear by the device 225. In some embodiments, the device 225 comprises a plurality of fenestrations 230. In some embodiments, adding the plurality of fenestrations 230 to the device 225 comprising a plurality of pores 231 improves egress of any subdermal fluid through the device and prevents subdermal fluid accumulation. FIG. 2C depicts a representation of the skin injury 212, wherein the skin flap is 100% viable after the device 225 was left in place for 2 weeks before removal.

The devices provided herein promote effective healing of the skin layers, epithelialization at the injured edge of the skin injury, and viability of separated skin tissue (e.g., a skin flap or a graft) by fixating and structurally supporting the skin injury during repair. The devices provided herein can reinforce or support repair of the skin injury. In some embodiments, the device can be affixed directly over the skin injury. In some embodiments, the scaffold can be affixed directly over the skin injury. In some embodiments, the device or the scaffold can provide multiplanar or multiaxial immobilization of the skin injury of the subject. In some embodiments, the device or the scaffold can prevent disruption of wound repair by immobilizing the skin injury of the subject. In some embodiments, the device or the scaffold can prevent disruption of wound repair due to shear forces, blistering, subdermal fluid accumulation, hematoma, sub graft fluid accumulation, or any combination thereof. In some embodiments, the device or the scaffold can provide reinforcement or support of wound repair. In some embodiments, the device or the scaffold can provide multiplanar or multiaxial reinforcement or support of wound repair.

In some embodiments, the device or the scaffold is configured to enclose or envelop the skin injury. In some embodiments, the device or the scaffold is configured to cover, enclose, or envelop at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the skin injury. In some embodiments, the device or the scaffold is configured to cover, enclose, or envelop about 20%, about 25%, about 30%, about 35%, about 40%, about 45%, about 50%, about 55%, about 60%, about 65%, about 70%, about 75%, about 80%, about 85%, about 90%, about 95%, or about 100% of the skin injury. In some embodiments, the device or the scaffold is configured to cover, enclose, or envelop at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the skin injury.

In some embodiments, the device or the scaffold is configured to cover, enclose, or envelop at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of an area of skin proximate to, surrounding, and comprising the skin injury. In some embodiments, the device or the scaffold is configured to cover, enclose, or envelop about 20%, about 25%, about 30%, about 35%, about 40%, about 45%, about 50%, about 55%, about 60%, about 65%, about 70%, about 75%, about 80%, about 85%, about 90%, about 95%, or about 100% of an area of skin proximate to, surrounding, and comprising the skin injury. In some embodiments, the device or the scaffold is configured to cover, enclose, or envelop at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of an area of skin proximate to, surrounding, and comprising the skin injury.

In some embodiments, the device or the scaffold is configured to cover, enclose, or envelop about 20% to about 100% of the skin injury of the subject. In some embodiments, the device or the scaffold is configured to cover, enclose, or envelop about 20% to about 30%, about 30% to about 40%, about 40% to about 50%, about 50% to about 60%, about 60% to about 70%, about 70% to about 80%, about 80% to about 90%, or about 90% to about 100% of the skin injury of the subject. In some embodiments, the device or the scaffold is configured to cover, enclose, or envelop about 20% to about 100% of an area of skin proximate to, surrounding, and comprising the skin injury. In some embodiments, the device or the scaffold is configured to cover, enclose, or envelop about 20% to about 30%, about 30% to about 40%, about 40% to about 50%, about 50% to about 60%, about 60% to about 70%, about 70% to about 80%, about 80% to about 90%, or about 90% to about 100% of an area of skin proximate to, surrounding, and comprising the skin injury.

The device can be configured to adhere to the skin injury of the subject without adhering to a covering (i.e., a dressing, textile, or article of clothing) over the device. In some embodiments, the second layer or the scaffold prevents at least one of a bandage, a compression bandage, a dressing, an absorbent pad, an absorbent material, a sleeve, a compression sleeve, a covering, a drape, a fabric, a textile, a gown, or an article of clothing contacting the device from adhering to the adhesive material (e.g., a first layer). In some embodiments, the device comprises a non-adherent overlay layer (e.g., a third layer).

Skin injury repair can be disrupted by removing a primary closure device before the skin injury has been sufficiently repaired (e.g., removed to replace the primary closure device, to drain fluid associated with wound injury or repair, to change an absorbent layer or a covering of the device, or to observe wound healing progress). Removing a primary closure device from a skin injury prematurely can disrupt wound repair, reinjure the skin injury, or result in repair failure. The devices described herein are configured to be continuously affixed to the skin injury throughout wound repair, preventing disruption of direct repair of the skin injury and promoting effective healing of skin layers, epithelialization at the injured edge of the skin injury, and viability of separated skin tissue. The devices described herein can be removed from the skin injury without disrupting skin injury repair or without injuring the subject or the skin injury.

In some embodiments, the device is configured to be affixed directly over the skin injury of the subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days. In some embodiments, the device is configured to be affixed directly over the skin injury of the subject for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the device is configured to be affixed directly over the skin injury of the subject for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days. In some embodiments, the device is configured to be affixed continuously for at least two days.

In some embodiments, the device is configured to be affixed directly over the skin injury of the subject for about 1 day to about 31 days. In some embodiments, the device is configured to be affixed directly over the skin injury of the subject for about 1 day to about 14 days. In some embodiments, the device is configured to be affixed directly over the skin injury of the subject for about 1 day to about 7 days, about 7 days to about 14 days, about 14 days to about 21 days, or about 21 days to about 31 days. In some embodiments, the device is configured to be affixed directly over the skin injury of the subject for about 5 days to about 14 days.

In some embodiments, the device is configured to remain affixed to the subject upon exposure to moisture from an environment. In some embodiments, the device is configured to be worn when the subject takes a shower or a bath.

A common strategy to mitigate risk of infection of a skin injury is to use an occlusive wound healing device or covering over the skin injury. An occlusive device prevents passage of air, moisture, fluid, or any combination thereof through the device. However, a repairing skin injury may generate fluid associated with injury or wound healing. A repairing skin injury must be kept clean, while draining or allowing for draining of fluid associated with injury or wound repair from the skin injury to prevent microbial growth and infection of the skin injury. In some embodiments, the devices described herein are configured to remain affixed directly over the skin injury, allowing the fluid to drain from the skin injury during wound healing or skin injury repair.

In some embodiments, the devices described herein are non-occlusive. Provided herein are non-occlusive devices configured to permit fluid egress from the skin injury, wherein the fluid can be associated with wound injury or repair. In some embodiments, the device is configured to permit egress of fluid from the skin injury without removing the device from the skin injury of the subject. In some embodiments, the first layer, the adhesive material, the second layer, the scaffold, a third layer, or any combination thereof can be non-occlusive. In some embodiments, the first layer, the adhesive material, the second layer, the scaffold, a third layer, or any combination thereof are configured to permit fluid egress from the skin injury, wherein the fluid can be associated with wound injury or repair. In some embodiments, the device comprises at least one a plurality of fenestrations or a plurality of pores configured to permit fluid egress from the skin injury without removing the device.

In some embodiments, while healing, a treated skin injury can exhibit a marker of healing, demonstrating progress of wound healing. In some embodiments, the device can be affixed continuously over the skin injury, and the devices and methods provided herein can allow for monitoring or observing a marker of healing without removing the device from the skin injury. In some embodiments, a marker of healing can be a cessation of draining of the wound. In some embodiments, the wound stops draining or producing fluid once epithelialization has occurred. In some embodiments, the wound will stop draining or producing fluid once the graft or skin flap is attached to the wound bed or the injured skin edge. In some embodiments, the marker of healing comprises a cessation of wound drainage from the skin injury, a cell layer of closure at an injured edge of the skin injury, an indicator of skin viability, blanching of the skin injury with pressure, pink coloration of the skin injury, or any combination thereof.

In some embodiments, the device is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% transparent. In some embodiments, the device is about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% transparent. In some embodiments, the device is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% transparent. In some embodiments, the device comprises a transparent material. In some embodiments, the adhesive material, the scaffold, a third layer, or any combination thereof comprises a transparent material. In some embodiments, adding a plurality of fenestrations to the device results in the device being at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% transparent. In some embodiments, a plurality of pores in the scaffold results in the device being at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% transparent. In some embodiments, a combination of a plurality of fenestrations and a plurality of pores makes the device at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% transparent.

### Staples and Sutures

Traumatic skin injuries or skin tears have an increased prevalence in elderly, critically ill/injured, or malnourished subjects. Some subjects, including elderly subjects, have fragile skin. Fragile skin increases the risk of the skin tearing due to shear, friction, or blunt forces. Moreover, the likelihood of skin flap survival in subjects with fragile skin is further decreased. Staples or sutures are not ideal to treat a skin injury in subjects with fragile skin because staples or sutures may further tear the skin. Skin injuries can also be caused by staples, sutures or the suture material tugging the edges of a skin injury. There is a need for a device that can provide primary closure of a skin injury without use of staples, sutures or suture material, thereby reducing the risk of further damage caused by stapling or suturing.

Provided herein are devices for treating a skin injury of a subject without staples, sutures or suture material. In some embodiments, the devices described herein are configured to provide primary closure of a skin injury without requiring use of staples, sutures or suture material to treat the skin injury. In some embodiments, the device is configured to affix to the subject without providing staples or sutures to the skin injury, the device, the subject, or any combination thereof. In some embodiments, deep tissue closure of the skin injury can be performed by the device. In some embodiments, both dermal and epidermal skin repair can be performed by the device.

However, the devices described herein are compatible with using staples, sutures or suture material. In some embodiments, staples or sutures provide “hybrid closure” with the devices described herein. In some embodiments, deep tissue closure of the skin injury can be performed by stapling or suturing and superficial skin repair can be performed by affixing the device directly over the skin injury of the subject. In some embodiments, deep dermal skin repair can be performed with staples or sutures and superficial skin repair can be performed with the device. In some embodiments, the staples or sutures can be absorbable. In some embodiments, the staples or sutures can be made with an absorbable material. In some embodiments, the suture material can be an absorbable material.

### Adhesives

The adhesive material should be durable and able to secure the scaffold in place even if wound drainage necessitates multiple dressing changes. The adhesive strength can be varied for a variety of applications. In some embodiments, the adhesive strength differs on at least two surfaces of the device, the first layer, the second layer, the scaffold, or any combination thereof.

In some embodiments, the first layer or the adhesive material is configured to affix the scaffold directly over the skin injury of the subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days. In some embodiments, the first layer or the adhesive material is configured to affix the scaffold directly over the skin injury of the subject for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the first layer or the adhesive material is configured to affix the scaffold directly over the skin injury of the subject for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days.

In some embodiments, the first layer or the adhesive material is configured to affix the scaffold directly over the skin injury of the subject for about 1 day to about 31 days. In some embodiments, the first layer or the adhesive material is configured to affix the scaffold directly over the skin injury of the subject for about 1 day to about 14 days. In some embodiments, the first layer or the adhesive material is configured to affix the scaffold directly over the skin injury of the subject for about 1 day to about 7 days, about 7 days to about 14 days, about 14 days to about 21 days, or about 21 days to about 31 days. In some embodiments, the first layer or the adhesive material is configured to affix the scaffold directly over the skin injury of the subject for about 5 days to about 14 days.

In some embodiments, the device is configured to removably attach to the skin injury via the adhesive material. In some embodiments, the adhesive material comprises a tape, a film, a glue, a spray, a gel, a foam, or any combination thereof. In some embodiments, the adhesive material is biocompatible. In some embodiments, the adhesive material is elastic. In some embodiments, the adhesive material is non-occlusive. In some embodiments, the first layer comprises the adhesive, wherein the first layer is non-occlusive.

### Non-Adhesive Backing

In some embodiments, the device comprises a non-adhesive backing layer attached to the adhesive material. In some embodiments, the backing layer is configured to be removed from the device, the first layer, or the adhesive material before affixing the second layer or the scaffold directly over the skin injury of the subject.

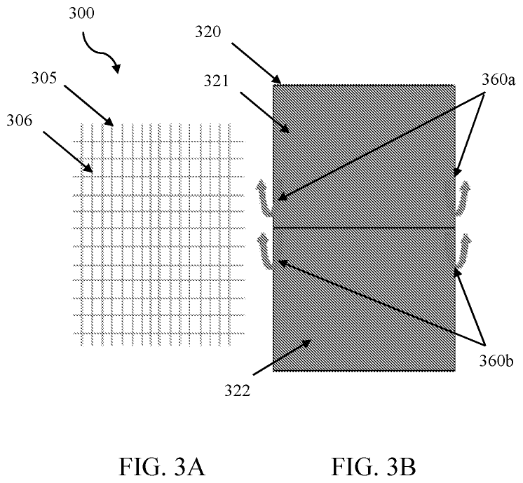

FIG. 3A depicts an exemplary device 300 of the present disclosure comprising a scaffold 305 coupled to an adhesive material 306, wherein the device 300 lacks a non-adhesive backing layer. FIG. 3B shows a non-adhesive backing layer 320 configured to removably adhere to the adhesive material 306 of the device 300. In some embodiments, the non-adhesive backing layer 320 comprises two pieces 321 and 322, wherein the backing layer 320 is configured to be peeled off the device before affixing the device to the subject by peeling away each of pieces 321 and 322. In some embodiments, one piece of the backing layer 321 can be removed first, then part of the device can be affixed to a skin flap and the skin flap can be pulled into anatomical position holding the device at the second piece 322 of non-adhesive backing layer 320, and then removing the second piece of non-adhesive backing layer 322 and affixing the rest of the device to the subject. The directional arrow pairs 360a and 360b in FIG. 3B indicate the directions of movement of each piece of non-adhesive backing in operation and the directional arrows form no part of the claimed device.

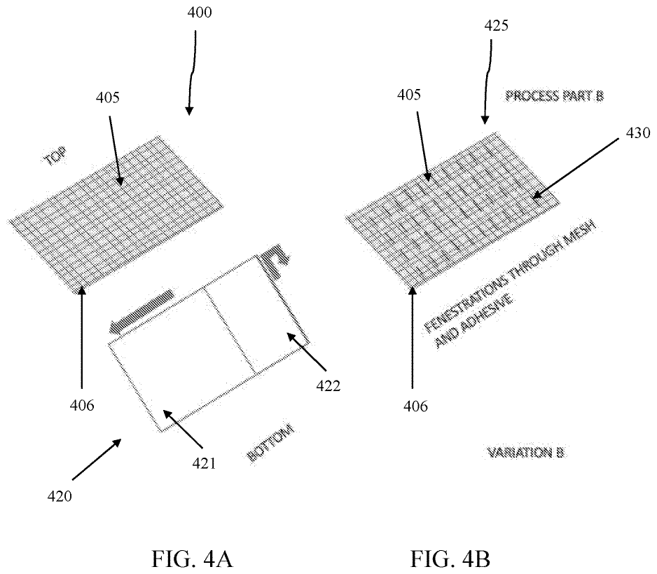

FIG. 4A depicts an exemplary device 400 of the present disclosure. The first layer or adhesive material 406 is coupled to the second layer or scaffold 405. A non-adhesive backing layer 420 can be attached to the adhesive material 406. The backing layer 420 comprises a first piece 421 and a second piece 422. In some embodiments, the first piece 421 and second piece 422 of the backing layer are the same size. In some embodiments, the first piece 421 and the second piece 422 of the backing layer can be different sizes. The pieces of the backing layer can be sized and configured to improve positioning and affixing the device directly over the skin injury. The directional arrows in FIG. 4A indicate the directions of movement of each piece of non-adhesive backing in operation and the directional arrows form no part of the claimed device.

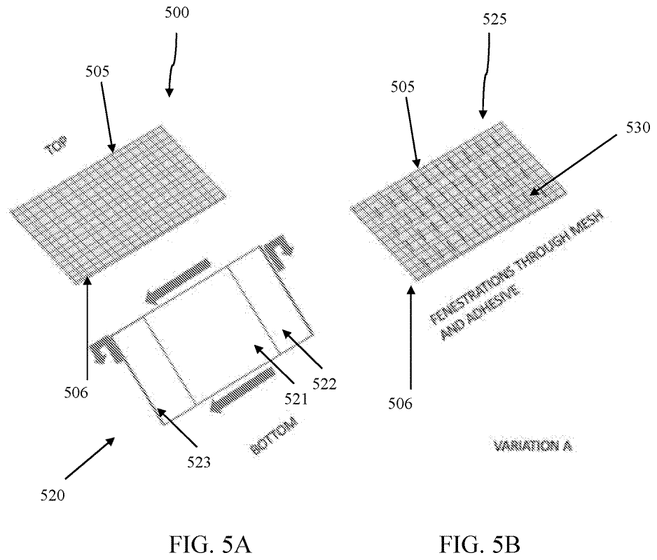

FIG. 5A depicts an exemplary device 500 of the present disclosure having a variation of the non-adhesive backing layer 520. In some embodiments, the adhesive material 506 coupled to the scaffold 505. A non-adhesive backing layer 520 can be removably attached to the adhesive material 506. In some embodiments, the backing layer 520 comprises a first piece 523, a second piece 522, and a third piece 521. In some embodiments, the first piece, the second piece, the third piece, or any combination thereof of the backing layer are the same size. In some embodiments, the first piece, the second piece, the third piece, or any combination thereof of the backing layer are different sizes. In some embodiments, the third piece 521 can be removed before the device is positioned to be affixed directly over the skin injury of the subject by a healthcare provider. In some embodiments, the healthcare provider positions the device 500 with the third piece 521 removed and affixes the adhesive material 506 and the scaffold 505 directly over a skin tear while holding the device 500 at the first and second pieces 521 and 522 (e.g., holding the “handle bars” to position the device before affixing the device to the subject). After the device is affixed to the skin tear, the first and second pieces 521 and 522 can be removed and the device 500 can be affixed to an area of skin of the subject directly adjacent to the skin tear. The directional arrows in FIG. 5A indicate the directions of movement of each piece of non-adhesive backing in operation and the directional arrows form no part of the claimed device.

### Fenestrations

In some embodiments, the device comprises a plurality of fenestrations. In some embodiments, the device comprises a plurality of fenestrations through a first layer, a second layer, a third layer, or any combination thereof. In some embodiments, a plurality of fenestrations can be added to a first layer, a second layer, a third layer, or any combination thereof. In some embodiments, the device comprises a plurality of fenestrations through the adhesive material, the scaffold, a non-adherent overlay layer, a non-adhesive backing layer, or any combination thereof. In some embodiments, a plurality of fenestrations can be added to the adhesive material, the scaffold, a non-adherent overlay layer, a non-adhesive backing layer, or any combination thereof. In some embodiments, a plurality of fenestrations facilitates wound drainage. In some embodiments, the plurality of fenestrations reduces the risk of infections of the skin injury due to plurality of fenestrations facilitating wound drainage.

FIG. 4B depicts an exemplary device of the present disclosure, wherein the device 400 became the nonocclusive device 425 when a plurality of fenestrations 430 was punctured through the adhesive material 406 and the scaffold 405. FIG. 5B depicts an exemplary device of the present disclosure, wherein the device 500 became the device 525 when a plurality of fenestrations 530 was punctured through the adhesive material 506 and the scaffold 505.

In some embodiments, adding fenestrations to the device, the adhesive material, the scaffold, a third layer, or any combination thereof results in the device being non-occlusive. In some embodiments, the plurality of fenestrations allows for egress of any fluid that develops from or adjacent to the skin injury, wherein the fluid can be associated with wound injury or repair. In some embodiments, the plurality of fenestrations is configured to permit egress of fluid from the skin injury associated with wound repair without removing the adhesive material, the first layer, the scaffold, the second layer, the device, or any combination thereof from the skin injury of the subject.

In some embodiments, the plurality of fenestrations is at least one of punched, cut, stamped, punctured, formed, sprayed, seared, burned, melted, or lasered through the first layer, the second layer, the third layer, or any combination thereof. In some embodiments, the plurality of fenestrations is at least one of punched, cut, stamped, punctured, formed, sprayed, seared, burned, melted, or lasered through the adhesive material, the scaffold, a non-adherent overlay layer, a non-adhesive backing layer, or any combination thereof.

In some embodiments, the device comprises a fenestration pattern. In some embodiments, the fenestration pattern is configured to preserve immobilization capacity or tensile strength of the device. In some embodiments, the fenestration pattern is configured to preserve immobilization capacity or tensile strength of at least one of the first layer, the adhesive material, the second layer, or the scaffold. In some embodiments, the fenestration pattern comprises a shape. In some embodiments, the fenestration pattern comprises at least one of a linear, a rectangular, a circular, an elliptical, a triangular, or a trapezoidal shape. In some embodiments, the fenestration pattern comprises at least one of a substantially linear, a substantially rectangular, a substantially circular, a substantially elliptical, a substantially triangular, or a substantially trapezoidal shape. The elongated sides of the shape can be wavy, straight, or any combination thereof (e.g., a substantially triangular shape comprises up to three wavy lines and has three angles). The elongated sides of the shape can have segments that are wavy, straight, or any combination thereof (e.g., in some embodiments, a substantially circular shape comprises an arc, wherein the arc comprises a wavy line or a point that is not equidistant from the center). In some embodiments, the fenestration pattern comprises a crossing pattern of at least two shapes (e.g., two linear shapes that cross to form an “X” or plus-shaped fenestration). In some embodiments, a fenestration pattern comprising a cross pattern or a crossing pattern allows for more fluid egress from the skin injury when the device is affixed directly over the skin injury.

In some embodiments, the fenestration pattern comprises a grid of shapes. In some embodiments, the grid of shapes comprises a plurality of linear shapes, a plurality of rectangular shapes, a plurality of circular shapes, a plurality of elliptical shapes, a plurality of triangular shapes, a plurality of trapezoidal shapes, or any combination thereof. In some embodiments, the grid of shapes comprises a plurality of substantially linear shapes, a plurality of substantially rectangular shapes, a plurality of substantially circular shapes, a plurality of substantially elliptical shapes, a plurality of substantially triangular shapes, a plurality of substantially trapezoidal shapes, or any combination thereof. In some embodiments, the grid of shapes comprises a crossing pattern of at least two shapes (e.g., a grid of two linear shapes crossed to form a plurality of “X” or plus-shaped fenestrations). In some embodiments, a grid of shapes comprising a crossing pattern allows for more fluid egress from the skin injury when the device is affixed directly over the skin injury.

The number of fenestrations, location of fenestrations, size of fenestrations, and fenestration pattern can be varied for different applications, greatly expanding the potential use of the device.

### Scaffold

Provided herein are devices comprising a scaffold, wherein the scaffold is configured to structurally support a skin injury of a subject. In some embodiments, the scaffold can reinforce or support repair of the skin injury of the subject. In some embodiments, the scaffold can be adaptable or configurable to complex wound shape and topography. In some embodiments, the scaffold can be flexible or elastic, allowing the device to conform to the shape of the wound site. In some embodiments, the scaffold provides tensile strength, durability, structure, elasticity, or any combination thereof to the device or the second layer of the device. In some embodiments, the scaffold bolsters or provides multiplanar or multiaxial immobilization of the skin injury of the subject. In some embodiments, the scaffold can provide multiplanar or multiaxial reinforcement of wound repair.

In some embodiments, the scaffold comprises a polymer, a nanofiber, a microfiber, a fiber, a metal, a wire, a string, a mesh, a fabric, a polyester, a textile, a foam, a gel foam, or any combination thereof. In some embodiments, the scaffold comprises a weave of polymers, nanofibers, microfibers, fibers, metals, wires, strings, meshes, fabrics, polyesters, textiles, foams, gel foams, or any combination thereof.

In some embodiments, the scaffold comprises a biocompatible material, a biologic, or any combination thereof. In some embodiments, the scaffold comprises a synthetic material. In some embodiments, the scaffold comprises an elastic material.

In some embodiments, the scaffold is non-occlusive. In some embodiments, the second layer is non-occlusive. In some embodiments, the scaffold is configured to permit egress of fluid from the skin injury associated with wound repair without removing the scaffold from the skin injury of the subject. In some embodiments, the scaffold comprises a plurality of pores. In some embodiments, the plurality of pores comprises an average pore size between 0.2 mm and 20 mm. In some embodiments, the average pore size is between about 0.2 mm to about 0.5 mm, about 0.5 mm to about 1.0 mm, about 1.0 mm to about 1.5 mm, or about 1.5 mm to about 2.0 mm. In some embodiments, the average pore size is about 0.2 mm, 0.4 mm, 0.6 mm, 0.8 mm, 1.0 mm, 1.2 mm, 1.4 mm, 1.6 mm, 1.8 mm, or 2.0 mm. In some embodiments, the plurality of pores allows for egress of any fluid that develops from or adjacent to the skin injury, wherein the fluid can be associated with wound injury or repair. In some embodiments, the plurality of pores is configured to permit egress of fluid from the skin injury associated with wound repair without removing the adhesive material, the first layer, the scaffold, the second layer, the device, or any combination thereof from the skin injury of the subject.

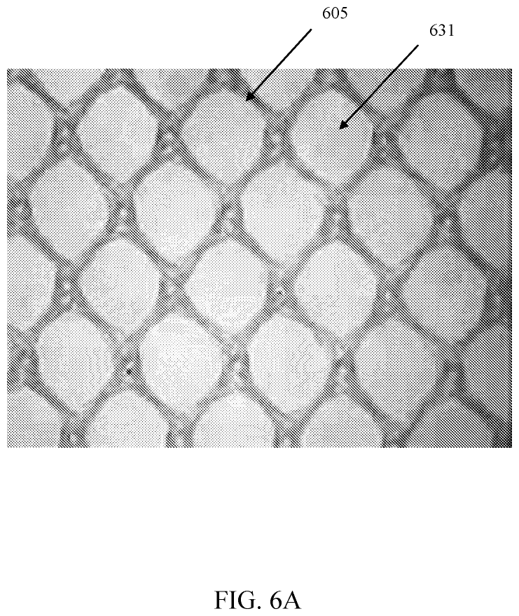

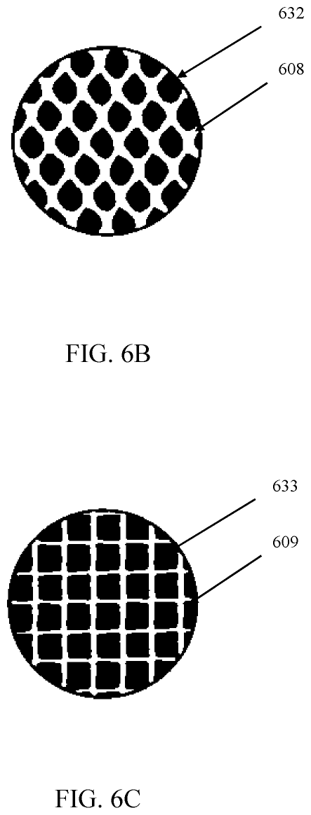

In some embodiments, the scaffold comprises a biocompatible mesh. FIG. 6A shows an exemplary scaffold of the present disclosure comprising a mesh 605, wherein the mesh 605 comprises a plurality of pores 631. FIGS. 6B and 6C depict an alternative exemplary scaffold of the present disclosure. FIG. 6B depicts a scaffold 608 comprising a plurality of circular pores 632. FIG. 6C depicts a scaffold 609 comprising a plurality of square-shaped pores 633.

In some embodiments, the scaffold comprises a polyester mesh. In some embodiments, the scaffold comprises a 30D polyester mesh. In some embodiments, the weight (g/m²) of the scaffold or mesh is between about 25 to about 45 g/m². In some embodiments, the thickness (mm) of the scaffold or mesh is between about 0.01 to about 3.0 mm. In some embodiments, the thickness (mm) of the scaffold or mesh is between about 0.15 to about 0.3 mm. The mesh scaffold can have a break strength in the machine direction (MD) of between about 120 Newtons (N) per 2.5 centimeters (cm) to about 862 N/2.5 cm. The mesh scaffold can have a break strength in the cross-machine direction (CMD) of between about 94 N/2.5 cm to about 497 N/2.5 cm. In some embodiments, the mesh scaffold can have a break strength (or grab strength) of between about 30 to about 220 N/2.5 cm. In some embodiments, the grab strength of the scaffold or mesh is between about 5 to about 50 pounds per inch (lb/in). In some embodiments, the elongation at break (%) of the scaffold or mesh is between about 20 to about 140 percent. In some embodiments, the Mullen Burst (kPA) of the scaffold or mesh is between about 50 to about 400 pKA. In some embodiments, the device can be manufactured as a large roll comprising at least one of the first layer, the second layer, a third layer, or the non-adhesive backing layer. The roll can be stored, and a piece can be cut from the roll, wherein the piece can be measured to fit the size of the skin injury of the subject. The piece from the roll comprises a device for treatment of a skin injury. The selected piece can be adaptable to complex wound shape and topography.

### Non-Adherent Overlay Layer

The adhesive material may frustrate removal or changing of an absorbent covering (e.g., a dressing). The adhesive material can be applied to the scaffold such that the adhesive material adheres the device to the skin injury but does not significantly adhere the device to the absorbent covering. In some embodiments, the scaffold or second layer is sufficiently non-adherent such that the device and the adhesive material (e.g., a first layer) can adhere to the skin injury without significantly adhering to an absorbent covering (e.g., dressing, textile, or article of clothing) placed over the device. A device comprising the adhesive material and the scaffold without a separate non-adherent overlay layer can be termed a “bilaminate device.” In some embodiments, a bilaminate device affixed directly over a skin injury of a subject does not adhere or minimally adheres to an absorbent covering.

In some cases, a non-adherent overlay layer (e.g., a third layer) can be applied to the device to prevent absorbent coverings from adhering to the device during routine changes of the coverings. If the device comprises an adhesive material (e.g., a first layer), a scaffold (e.g., a second layer), and a non-adherent overlay layer, it can be termed a “trilaminate device.” In some embodiments, a trilaminate device affixed directly over a skin injury of a subject does not adhere or minimally adheres to an absorbent covering.

In some embodiments, the device comprises a third layer configured to prevent at least one of a bandage, a compression bandage, a dressing, an absorbent pad, an absorbent material, a sleeve, a compression sleeve, a covering, a drape, a fabric, a textile, a gown, or an article of clothing contacting the device from adhering or affixing to the device. In some embodiments, the third layer is non-adhesive. In some embodiments, the third layer can be coupled to at least one of the first layer or the second layer. In some embodiments, the third layer can be coupled to at least one of the adhesive material or the scaffold. In some embodiments, the third layer comprises a non-adhesive overlay, material, surface, or any combination thereof. In some embodiments, at least one of the third layer, the non-adhesive overlay, the non-adhesive material, or the non-adhesive surface comprises a tape, a film, a glue, a spray, a gel, a foam, or any combination thereof. In some embodiments, the third layer is non-occlusive.

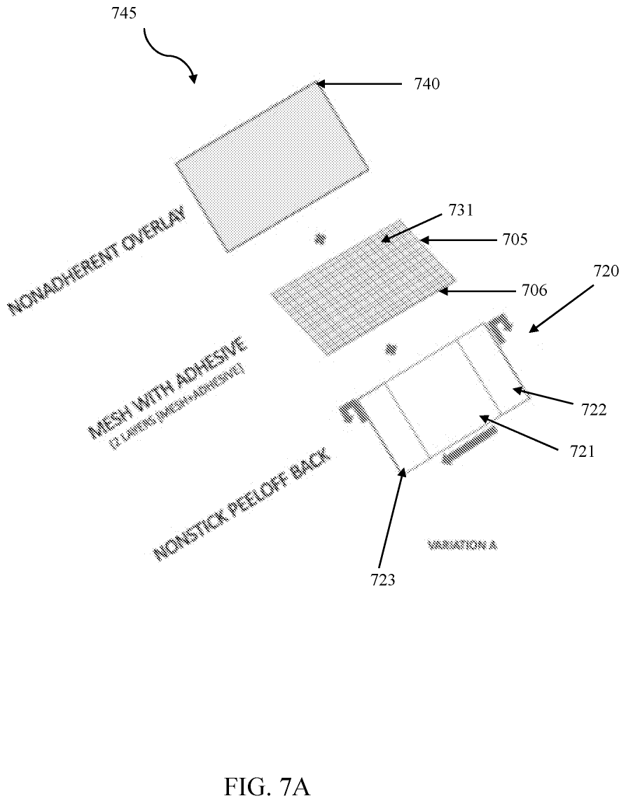

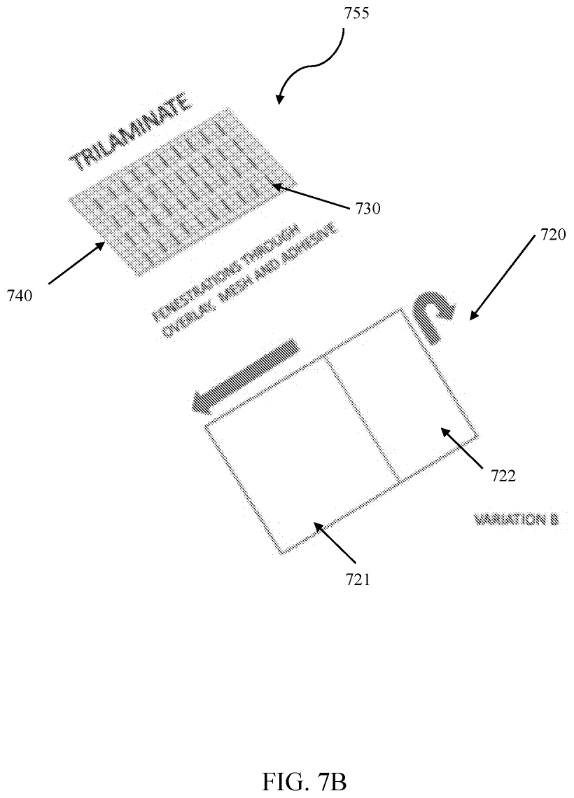

FIG. 7A depicts an exemplary trilaminate device 745 of the present disclosure. In some embodiments, the device 745 comprises a scaffold 705 and an adhesive material 706. In some embodiments, the scaffold 705 comprises a plurality of pores 731. In some embodiments, a non-adherent overlay 740 can be attached to the scaffold 705. In some embodiments, the non-adherent overlay 740 prevents clothing the subject is wearing from adhering or at least partially adhering to the device 745. In some embodiments, the non-adherent overlay 740 prevents a bandage or dressing covering the device 745 from adhering or at least partially adhering to the device 745. In some embodiments, a non-adhesive backing layer 720 can be attached to the adhesive material 706, wherein the backing layer 720 comprises a variation having three pieces 721, 722, and 723.

FIG. 7B depicts a plurality of fenestrations 730 through a trilaminate device 755. In some embodiments, the trilaminate device 755 comprises a non-adherent overlay 740 attached to the scaffold and adhesive material, wherein all three layers comprise the plurality of fenestrations 730. In some embodiments, the trilaminate device 755 further comprises a non-adhesive backing layer 720 attached to the adhesive material. In some embodiments, the non-adhesive backing layer 720 is a variation comprising two pieces 721 and 722.

### Free Edge

In some embodiments, when the skin injury is sufficiently healed, the device can be removed by the subject or a healthcare provider. In some embodiments, the device can be affixed to the subject. In some embodiments, the device comprises a “free edge” to facilitate removal of the device. In some embodiments, the “free edge” comprises a surface configured to contact the subject without affixing to the subject. In some embodiments, the free edge can be configured to have less adhesive strength than the adhesive strength between the scaffold and the skin injury of the subject.

In some embodiments, the free edge of the device does not comprise an adhesive material. In some embodiments, the device comprises a surface that is configured to contact the subject without affixing to the subject for at least 2 days. In some embodiments, the first layer or the second layer comprises a surface that is configured to contact the subject without affixing to the subject for at least 2 days. In some embodiments, the scaffold comprises a surface that is configured to contact the subject without affixing to the subject for at least 2 days.

In some embodiments, the first layer can be partially applied, minimally applied, absent, partially removed, or fully removed from a surface of the second layer, and wherein the surface of the second layer is configured to contact the subject. In some embodiments, the first layer or the adhesive material can be partially applied, minimally applied, absent, partially removed, or fully removed from a surface of the second layer or the scaffold, and wherein the surface of the second layer or the scaffold is configured to contact the subject. In some embodiments, the surface of the second layer or the scaffold is the “free edge.”

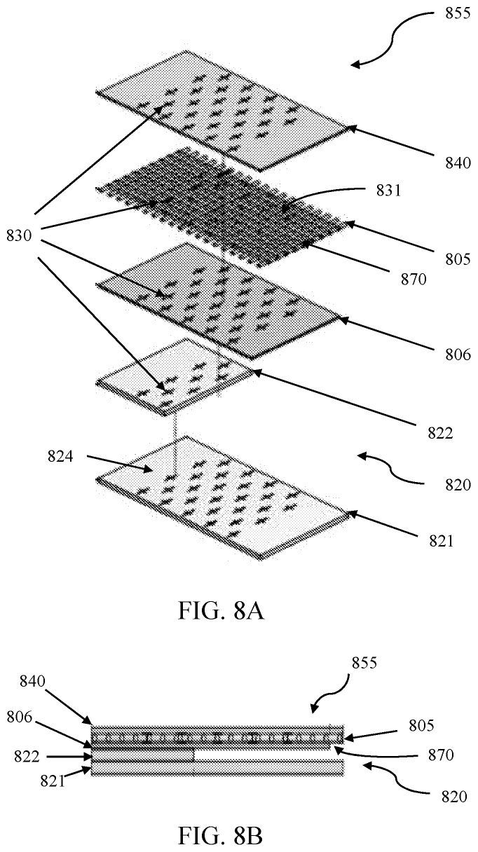

FIGS. 8A-8B depict perspective exploded and elevational views, respectively, of an exemplary non-occlusive trilaminate device 855 of the present disclosure comprising a free edge 870. In some embodiments, a scaffold 805 comprises the free edge 870. A side of the scaffold 805 configured to contact a skin injury of a subject can be coupled to an adhesive material layer 806, wherein the adhesive material layer 806 is not applied to the free edge 870. The free edge 870 is configured to contact the subject without affixing to the subject. In some embodiments, the scaffold 805 further comprises a plurality of pores 831. In some embodiments, a non-adherent overlay layer 840 is coupled to a side of the scaffold 805 configured to contact a wound covering or bandage and is configured to prevent the non-occlusive trilaminate device 855 from adhering to the wound covering or bandage. In some embodiments, the non-adherent overlay layer 840 is at least about 50% transparent. In some embodiments, the non-occlusive trilaminate device 855 comprises a plurality of fenestrations 830 through the non-adherent overlay layer 840, the scaffold 805, and the adhesive material layer 806. In some embodiments, the non-occlusive trilaminate device 855 comprises a non-adhesive backing layer 820 removably attached to the adhesive material layer 806. In some embodiments, the non-adhesive backing layer 820 can be attached to the adhesive material layer 806 when the plurality of fenestrations 830 are added to the non-adherent overlay layer 840, the scaffold 805, the adhesive material layer 806, or any combination thereof.

In some embodiments, the non-adhesive backing layer 820 comprises two pieces, an outer piece 821 and an inner piece 822. In some embodiments, the two pieces 821 and 822 partially overlap and contact at a surface 824. The two pieces 821 and 822 being overlapped can assist a user of the device in removing the outer piece 821 from the non-occlusive device 855 before affixing the device directly over the skin injury of the subject. With the outer piece 821 removed, the user can hold the device at the inner piece 822 and position the free edge 870 relative to the skin injury before affixing the device directly over the skin injury of the subject (e.g., positioning the free edge away from the leading edge of the skin injury to prevent reinjury at the leading edge of the skin injury when removing the device using the free edge). After affixing a portion of the device 855 to the subject, the person using the device can remove the inner piece 822 and affix the rest of the affixable area of the device 855 to the subject.

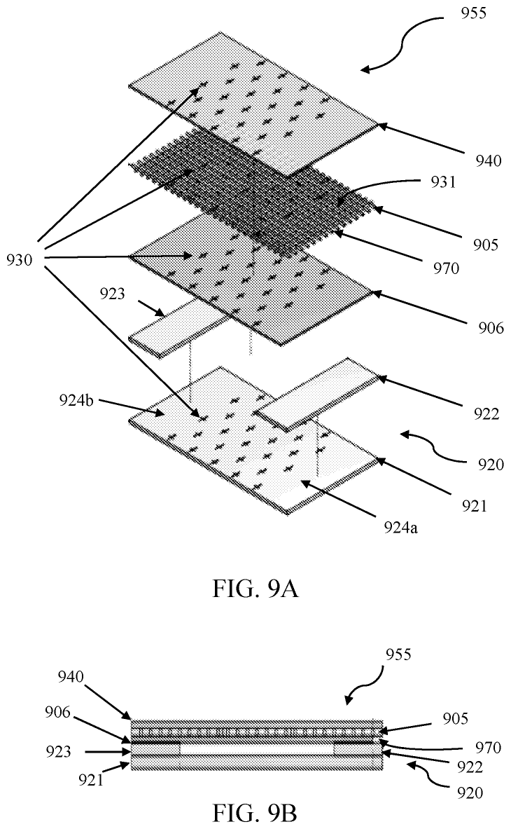

FIGS. 9A-9B depict perspective exploded and elevational views, respectively, of an exemplary non-occlusive trilaminate device 955 of the present disclosure comprising a free edge 970 and a “handle bar” type of non-adhesive backing layer. In some embodiments, a scaffold 905 comprises a free edge 970. A side of the scaffold 905 configured to contact a skin injury of a subject can be coupled to an adhesive material layer 906, wherein the adhesive material layer 906 is not applied to the free edge 970. The free edge 970 is configured to contact the subject without affixing to the subject. In some embodiments, the scaffold 905 further comprises a plurality of pores 931. In some embodiments, a non-adherent overlay layer 940 can be coupled to a side of the scaffold 905 configured to contact a wound covering or bandage and is configured to prevent the non-occlusive trilaminate device 955 from adhering to the wound covering or bandage. In some embodiments, the non-adherent overlay layer 940 is at least about 50% transparent. In some embodiments, the non-occlusive trilaminate device comprises a plurality of fenestrations 930 through the non-adherent overlay layer 940, the scaffold 905, and the adhesive material layer 906. In some embodiments, the non-occlusive trilaminate device 955 comprises a non-adhesive backing layer 920 removably attached to the adhesive material layer 906. In some embodiments, the non-adhesive backing layer 920 can be attached to the adhesive material layer 906 when the plurality of fenestrations 930 are added to the non-adherent overlay layer 940, the scaffold 905, and the adhesive material layer 906.

In some embodiments, the non-adhesive backing layer 920 comprises three pieces, outer piece 921, and handle bar pieces 922 and 923. In some embodiments, the outer piece 921 and handle bar piece 922 partially overlap and contact at a surface 924a. In some embodiments, the outer piece 921 and handle bar piece 923 partially overlap and contact at a surface 924b. Overlapping the outer piece 921 with one or both of the handle bar pieces 922 and 923 can assist a user in removing the outer piece 921 from the non-occlusive trilaminate device 955 before affixing the device directly over the skin injury of the subject. In some embodiments, with the outer piece 921 removed, the person using the device can hold the device at the handle bar pieces 922 and 923 (e.g., hold the device by its “handle bars”) and position the free edge 970 relative to the skin injury before affixing the device 955 directly over the skin injury of the subject (e.g., positioning the free edge away from the leading edge of the skin injury to prevent reinjury at the leading edge of the skin injury when removing the device using the free edge). In some embodiments, after affixing a portion of the device 955 to the subject, a user (e.g., an individual or a provider) using the device can remove the handle bar pieces 922 and 923 and affix the rest of the adhesive material layer 906 to the subject. A non-adhesive backing layer comprising three pieces can be particularly effective when treating larger-area skin injuries by affixing sheets (i.e., large pieces) of the non-occlusive device of the present disclosure.

### Removal

In some embodiments, the device is configured to be removed from the skin injury of the subject when the skin injury exhibits a marker of healing, the skin injury is attached to a wound bed of the subject, or any combination thereof. The device can be removed without causing additional injury to the subject or skin injury once the skin injury exhibits a marker of healing and/or the skin injury is attached or at least partially attached to the wound bed of the subject.

In some embodiments, the device or scaffold is configured to be removed from the skin injury immediately or after at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 12 minutes, at least about 13 minutes, at least about 14 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days from when the skin injury exhibits a marker of healing. In some embodiments, the device or scaffold is configured to be removed from the skin injury immediately or after about 1 minute, about 2 minutes, about 3 minutes, about 4 minutes, about 5 minutes, about 6 minutes, about 7 minutes, about 8 minutes, about 9 minutes, about 10 minutes, about 12 minutes, about 13 minutes, about 14 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days from when the skin injury exhibits a marker of healing. In some embodiments, the device or scaffold is configured to be removed from the skin injury immediately or after at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 13 minutes, at least 14 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days from when the skin injury exhibits a marker of healing.

In some embodiments, the device or scaffold is configured to be removed from the skin injury after about 1 day to about 31 days. In some embodiments, the device or scaffold is configured to be removed from the skin injury after about 1 day to about 14 days. In some embodiments, the device or scaffold is configured to be removed from the skin injury after about 1 day to about 7 days, about 7 days to about 14 days, about 14 days to about 21 days, or about 21 days to about 31 days. In some embodiments, the device or scaffold is configured to be removed from the skin injury after about 5 days to about 14 days.

### Methods

Also provided herein are methods for treating a skin injury of a subject, comprising: a) affixing a non-occlusive device directly over the skin injury of the subject, wherein the non-occlusive device comprises: (i) a first layer comprising an adhesive material; and (ii) a second layer comprising a scaffold configured to structurally support the skin injury of the subject, wherein the first layer is configured to affix the second layer directly over the skin injury of the subject. Also provided herein are methods for treating a skin injury of a subject, comprising: a) applying an adhesive material to the subject; and b) affixing a non-occlusive device directly over the skin injury of the subject, wherein the non-occlusive device comprises a scaffold configured to structurally support the skin injury, wherein the adhesive material is configured to affix the device directly over the skin injury of the subject. In some embodiments, the scaffold is non-occlusive. In some embodiments, the method comprises coupling the first layer or the adhesive material to the second layer or the scaffold.

The methods provided herein promote effective healing of the skin layers, epithelialization at the injured edge of the skin injury, and viability of separated skin tissue (e.g., a skin flap or a graft) by fixating and structurally supporting the skin injury during repair. In some embodiments, the methods provided herein can reinforce or support repair of the skin injury. In some embodiments, the device can be affixed directly over the skin injury. In some embodiments, the scaffold can be affixed directly over the skin injury. In some embodiments, the device or the scaffold can provide multiplanar or multiaxial immobilization of the skin injury of the subject. In some embodiments, the device or the scaffold can prevent disruption of wound repair by immobilizing the skin injury of the subject. In some embodiments, the device or the scaffold can prevent disruption of wound repair due to shear forces, blistering, subdermal fluid accumulation, hematoma, sub graft fluid accumulation, or any combination thereof. In some embodiments, the method comprises providing multiplanar or multiaxial immobilization of the skin injury of the subject with the device or the scaffold. In some embodiments, the method comprises reinforcing or supporting wound repair by providing multiplanar or multiaxial immobilization of the skin injury. In some embodiments, the method comprises preventing disruption of wound repair by immobilizing the skin injury of the subject with the device or the scaffold. In some embodiments, method comprises preventing disruption of wound repair due to shear forces, blistering, subdermal fluid accumulation, hematoma, sub graft fluid accumulation, or any combination thereof. In some embodiments, the method comprises providing multiplanar or multiaxial immobilization to the graft with the device or the scaffold. In some embodiments, the method comprises preventing shear forces, blistering, subdermal fluid accumulation, hematoma, sub graft fluid accumulation, or any combination thereof from disrupting attachment of the graft to a wound bed of the subject with the device or the scaffold.

In some embodiments, the device or the scaffold is configured to enclose or envelop the skin injury. In some embodiments, the device or the scaffold is configured to cover, enclose, or envelop at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the skin injury. In some embodiments, the device or the scaffold is configured to cover, enclose, or envelop about 20%, about 25%, about 30%, about 35%, about 40%, about 45%, about 50%, about 55%, about 60%, about 65%, about 70%, about 75%, about 80%, about 85%, about 90%, about 95%, or about 100% of the skin injury. In some embodiments, the device or the scaffold is configured to cover, enclose, or envelop at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the skin injury.

In some embodiments, the device or the scaffold is configured to cover, enclose, or envelop at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of an area of skin proximate to, surrounding, and comprising the skin injury. In some embodiments, the device or the scaffold is configured to cover, enclose, or envelop about 20%, about 25%, about 30%, about 35%, about 40%, about 45%, about 50%, about 55%, about 60%, about 65%, about 70%, about 75%, about 80%, about 85%, about 90%, about 95%, or about 100% of an area of skin proximate to, surrounding, and comprising the skin injury. In some embodiments, the device or the scaffold is configured to cover, enclose, or envelop at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of an area of skin proximate to, surrounding, and comprising the skin injury.

In some embodiments, the method comprises covering, enclosing, or enveloping at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the skin injury or an area of skin proximate to, surrounding, and comprising the skin injury with the device or the scaffold.

In some embodiments, the device is configured to be affixed directly over the skin injury of the subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days. In some embodiments, the device is configured to be affixed directly over the skin injury of the subject for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the device is configured to be affixed directly over the skin injury of the subject for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days. In some embodiments, the device is configured to be affixed continuously for at least two days.

In some embodiments, the device is configured to be affixed directly over the skin injury of the subject for about 1 day to about 31 days. In some embodiments, the device is configured to be affixed directly over the skin injury of the subject for about 1 day to about 14 days. In some embodiments, the device is configured to be affixed directly over the skin injury of the subject for about 1 day to about 7 days, about 7 days to about 14 days, about 14 days to about 21 days, or about 21 days to about 31 days. In some embodiments, the device is configured to be affixed directly over the skin injury of the subject for about 5 days to about 14 days.

In some embodiments, the device is configured to remain affixed to the subject upon exposure to moisture from an environment. In some embodiments, the device is configured to be worn when the subject takes a shower or a bath.

In some embodiments, the method comprises affixing the device directly over the skin injury of the subject continuously for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days. In some embodiments, the method comprises affixing the device directly over the skin injury of the subject continuously for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the method comprises affixing the device directly over the skin injury of the subject continuously for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days.

In some embodiments, the method comprises affixing the device directly over the skin injury of the subject continuously for about 1 day to about 31 days. In some embodiments, the method comprises affixing the device directly over the skin injury of the subject continuously for about 1 day to about 14 days. In some embodiments, the method comprises affixing the device directly over the skin injury of the subject continuously for about 1 day to about 7 days, about 7 days to about 14 days, about 14 days to about 21 days, or about 21 days to about 31 days. In some embodiments, the method comprises affixing the device directly over the skin injury of the subject continuously for about 5 days to about 14 days.

In some embodiments, the methods described herein comprise a non-occlusive device. Provided herein are non-occlusive devices configured to permit fluid egress from the skin injury, wherein the fluid can be associated with wound injury or repair. In some embodiments, the device is configured to permit egress of fluid from the skin injury without removing the device from the skin injury of the subject. In some embodiments, the first layer, the adhesive material, the second layer, the scaffold, a third layer, or any combination thereof are configured to permit fluid egress from the skin injury, wherein the fluid is associated with wound injury or repair. In some embodiments, the device comprises at least one a plurality of fenestrations or a plurality of pores configured to permit fluid egress from the skin injury without removing the device.

In some embodiments, the device is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% transparent. In some embodiments, the device is about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% transparent. In some embodiments, the device is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% transparent. In some embodiments, the device comprises a transparent material. In some embodiments, the adhesive material, the scaffold, a third layer, or any combination thereof comprises a transparent material. In some embodiments, adding a plurality of fenestrations to the device results in the device being at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% transparent. In some embodiments, a plurality of pores in the scaffold results in the device being at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% transparent. In some embodiments, a combination of a plurality of fenestrations and a plurality of pores makes the device at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% transparent. In some embodiments, the method comprises monitoring or observing a marker of healing without removing the device from the skin injury. In some embodiments, the method comprises monitoring or observing direct repair of the skin injury without removing the device from the skin injury.

### Method of Covering

In some embodiments, the method comprises covering the device with an absorbent dressing, wherein the dressing comprises at least one of a bandage, gauze, pad, absorbent pad, sleeve, compression sleeve, compression bandage, wound covering, fabric, or textile. In some embodiments, the method comprises changing the absorbent dressing without removing, changing, or displacing the device. In some embodiments, the method comprises permitting fluid from the skin injury of the subject associated with wound repair to pass through the device to the dressing. The absorbent dressing is removable without removing, changing, or displacing the devices described herein (e.g., the scaffold remains affixed directly over the skin injury when the absorbent dressing is changed). The method can comprise affixing a bilaminate or trilaminate device directly over the skin injury, wherein the bilaminate or trilaminate device does not adhere to the absorbent dressing.

### Staples and Sutures

Provided herein are methods for treating a skin injury of a subject without staples, sutures or suture material. In some embodiments, the methods described herein are configured to provide primary closure of a skin injury without requiring use of staples, sutures or suture material to treat the skin injury. In some embodiments, the device is configured to affix to the subject without providing staples or sutures to the skin injury, the device, the subject, or any combination thereof. In some embodiments, deep tissue closure of the skin injury can be performed by the device. In some embodiments, dermal and/or epidermal skin repair can be performed by the device.

The methods described herein are compatible with using staples, sutures or suture material. In some embodiments, staples or sutures provide “hybrid closure” with the methods described herein. In some embodiments, the method comprises providing staples or sutures to at least one of the skin injury, a skin layer, a deep tissue, the subcutis, the dermis, or the epidermis of the subject before affixing the device directly over the skin injury of the subject. In some embodiments, the method comprises performing deep tissue closure with staples or sutures before affixing the device directly over the skin injury of the subject. In some embodiments, the method comprises providing staples or sutures to the device, the subject, the skin injury, a skin layer of the subject, or any combination thereof after affixing the device directly over the skin injury of the subject. In some embodiments, the staples or sutures comprise an absorbable material. In some embodiments, the suture material can be an absorbable material. In some embodiments, the method comprises providing absorbable staples or sutures to at least one of the skin injury, a skin layer, a deep tissue, the subcutis, the dermis, or the epidermis of the subject before affixing the device directly over the skin injury of the subject.

In some embodiments, deep tissue closure of the skin injury can be performed by stapling or suturing and superficial skin repair can be performed by affixing the device directly over the skin injury of the subject. In some embodiments, deep dermal skin repair can be performed with staples or sutures and superficial skin repair can be performed with the device.

### Adhesives

In some embodiments, the first layer or the adhesive material is configured to affix the scaffold directly over the skin injury of the subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days. In some embodiments, the first layer or the adhesive material is configured to affix the scaffold directly over the skin injury of the subject for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the first layer or the adhesive material is configured to affix the scaffold directly over the skin injury of the subject for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days.

In some embodiments, the first layer or the adhesive material is configured to affix the scaffold directly over the skin injury of the subject for about 1 day to about 31 days. In some embodiments, the first layer or the adhesive material is configured to affix the scaffold directly over the skin injury of the subject for about 1 day to about 14 days. In some embodiments, the first layer or the adhesive material is configured to affix the scaffold directly over the skin injury of the subject for about 1 day to about 7 days, about 7 days to about 14 days, about 14 days to about 21 days, or about 21 days to about 31 days. In some embodiments, the first layer or the adhesive material is configured to affix the scaffold directly over the skin injury of the subject for about 5 days to about 14 days.

In some embodiments, the device is configured to removably attach to the skin injury via the adhesive material. In some embodiments, the adhesive material comprises a tape, a film, a glue, a spray, a gel, a foam, or any combination thereof. In some embodiments, the adhesive material is biocompatible. In some embodiments, the adhesive material is elastic. In some embodiments, the adhesive material is non-occlusive. In some embodiments, the method comprises removing the device without injuring the subject or the skin injury.

In some embodiments, the method comprises affixing the second layer or the scaffold directly over the skin injury of the subject continuously for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days. In some embodiments, the method comprises affixing the second layer or the scaffold directly over the skin injury of the subject continuously for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the method comprises affixing the second layer or the scaffold directly over the skin injury of the subject continuously for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days. In some embodiments, the method comprises affixing the scaffold directly over the skin injury of the subject using an adhesive material comprising a tape, a film, a glue, a spray, a gel, a foam, or any combination thereof. In some embodiments, the adhesive material can be applied to the subject, the skin injury, the device, or any combination thereof. In some embodiments, the method comprises applying the adhesive material to the device or the scaffold before the device or the scaffold is affixed directly over the skin injury of the subject. In some embodiments, the method comprises applying the adhesive material to the subject before the device or the scaffold is affixed directly over the skin injury of the subject. In some embodiments, the method comprises applying an additional adhesive material to the subject, the skin injury, or the device before the device or the scaffold is affixed directly over the skin injury of the subject.

In some embodiments, the method comprises affixing the second layer or the scaffold directly over the skin injury of the subject continuously for about 1 day to about 31 days. In some embodiments, the method comprises affixing the second layer or the scaffold directly over the skin injury of the subject continuously for about 1 day to about 14 days. In some embodiments, the method comprises affixing the second layer or the scaffold directly over the skin injury of the subject continuously for about 1 day to about 7 days, about 7 days to about 14 days, about 14 days to about 21 days, or about 21 days to about 31 days. In some embodiments, the method comprises affixing the second layer or the scaffold directly over the skin injury of the subject continuously for about 5 days to about 14 days.

### Non-Adhesive Backing

In some embodiments, the method comprises removing a non-adhesive backing layer attached to the adhesive material before affixing the non-occlusive device directly over the skin injury of the subject. In some embodiments, the method comprises a user (e.g., a healthcare provider or the subject) holding the device at one or more pieces of the backing layer when affixing the device directly over the skin injury of the subject.

### Fenestrations

In some embodiments, the method comprises adding a plurality of fenestrations or a fenestration pattern to the adhesive material, the scaffold, a third layer, or any combination thereof. In some embodiments, the method comprises adding a plurality of fenestrations or a fenestration pattern to the first layer, the second layer, the third layer, or any combination thereof. In some embodiments, the method comprises adding a plurality of fenestrations or a fenestration pattern to the adhesive material, the first layer, the scaffold, the second layer, the third layer, or any combination thereof. Fenestrations may be added to the scaffold while preserving immobilization capacity, tensile strength, durability, structure, elasticity, or any combination thereof of the scaffold.

### Non-Adherent Overlay Layer

In some embodiments, the method comprises preventing at least one of a bandage, a compression bandage, a dressing, an absorbent pad, an absorbent material, a sleeve, a compression sleeve, a covering, a drape, a fabric, a textile, a gown, or an article of clothing contacting the device from adhering or at least partially adhering to the device. In some embodiments, the method comprises affixing a bilaminate device directly over the skin injury of the subject, wherein the bilaminate device does not adhere or minimally adheres to a wound dressing covering the device. In some embodiments, the method comprises affixing a device comprising a non-adherent overlay layer (e.g., third layer) directly over the skin injury of the subject.

In some embodiments, the device comprises a non-adherent overlay layer to prevent at least one of a bandage, a compression bandage, a dressing, an absorbent pad, an absorbent material, a sleeve, a compression sleeve, a covering, a drape, a fabric, a textile, a gown, or an article of clothing contacting the device from adhering or at least partially adhering to the device. In some embodiments, the method comprises preventing at least one of a bandage, a compression bandage, a dressing, an absorbent pad, an absorbent material, a sleeve, a compression sleeve, a covering, a drape, a fabric, a textile, a gown, or an article of clothing contacting the device from adhering or at least partially adhering to the device by applying a non-adherent overlay layer to the device.

### Free Edge

In some embodiments, the method comprises removing the device from the skin injury of the subject via a free edge of the device. In some embodiments, the adhesive material can be removed or absent from the free edge. In some embodiments, the adhesive material can be minimally or partially applied to the free edge. In some embodiments, the method comprises removing the device in a directionality relative to the skin injury that prevents reinjury of the skin injury (e.g., peeling off the device in a direction that moves towards the leading edge of a healed skin injury). In some embodiments, the method comprises orienting or positioning a free edge of the device relative to the skin injury before affixing the device directly over the skin injury of the subject (e.g., positioning the free edge away from the leading edge of the skin injury to prevent reinjury at the leading edge of the skin injury when removing the device using the free edge).

### Removal

In some embodiments, the method comprises removing the device or scaffold from the skin injury of the subject when the skin injury exhibits a marker of healing, the skin injury is attached to a wound bed of the subject, or any combination thereof. In some embodiments, the method comprises removing the device from the skin injury immediately or after at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 12 minutes, at least about 13 minutes, at least about 14 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days from when the skin injury exhibits a marker of healing. In some embodiments, the method comprises removing the device or scaffold from the skin injury of the subject when the skin injury exhibits a marker of healing, the skin injury is attached to a wound bed of the subject, or any combination thereof. In some embodiments, the method comprises removing the device from the skin injury immediately or after about 1 minute, about 2 minutes, about 3 minutes, about 4 minutes, about 5 minutes, about 6 minutes, about 7 minutes, about 8 minutes, about 9 minutes, about 10 minutes, about 12 minutes, about 13 minutes, about 14 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days from when the skin injury exhibits a marker of healing. In some embodiments, the method comprises removing the device or scaffold from the skin injury of the subject when the skin injury exhibits a marker of healing, the skin injury is attached to a wound bed of the subject, or any combination thereof. In some embodiments, the method comprises removing the device from the skin injury immediately or after at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 13 minutes, at least 14 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days from when the skin injury exhibits a marker of healing.

In some embodiments, the method comprises removing the device or scaffold from the skin injury of the subject after about 1 day to about 31 days from when it is affixed to the skin injury of the subject. In some embodiments, the method comprises removing the device or scaffold from the skin injury of the subject after about 1 day to about 14 days from when it is affixed to the skin injury of the subject. In some embodiments, the method comprises removing the device or scaffold from the skin injury of the subject after about 1 day to about 7 days, about 7 days to about 14 days, about 14 days to about 21 days, or about 21 days to about 31 days from when it is affixed to the skin injury of the subject. In some embodiments, the method comprises removing the device or scaffold from the skin injury of the subject after about 5 days to about 14 days from when it is affixed to the skin injury of the subject.

In some embodiments, the method comprises removing the device from the skin injury once the skin injury exhibits a marker of healing, the skin injury is attached or at least partially attached to a wound bed of the subject, or any combination thereof, wherein removal of the device does not disrupt wound repair, reinjure the skin injury, detach the skin injury from the wound bed, or result in repair failure.

In some embodiments, the method comprises removing the device from the skin injury after seven days without disrupting wound repair, reinjuring the skin injury, detaching the skin injury from the wound bed, or resulting in repair failure.

### Additional Treatments

In some embodiments, the device comprises at least one of an antimicrobial, a silver antimicrobial, a silver material, an antimicrobial gel foam, an active ingredient, an antibiotic, an antifungal, a wound care product, a biologic, a medicament, an antiseptic, an anesthetic, an analgesic, a therapeutic, a hemostatic agent, or any combination thereof. In some embodiments, the first layer, the adhesive material, the second layer, the scaffold, or any combination thereof, comprises at least one of an antimicrobial, a silver antimicrobial, a silver material, an antimicrobial gel foam, an active ingredient, an antibiotic, an antifungal, a wound care product, a biologic, a medicament, an antiseptic, an anesthetic, an analgesic, a therapeutic, a hemostatic agent, or any combination thereof.

In some embodiments, the method comprises providing an antimicrobial, a silver antimicrobial, a silver material, an antimicrobial gel foam, an active ingredient, an antibiotic, an antifungal, a wound care product, a biologic, a medicament, an antiseptic, an anesthetic, an analgesic, a therapeutic agent, a hemostatic agent, or any combination thereof to or adjacent to the skin injury of the subject.

In some embodiments, the antimicrobial comprises at least one of an over-the-counter antimicrobial treatment, a prescribed antimicrobial treatment, silver, a silver antimicrobial, or a gel foam material with antimicrobial properties. In some embodiments, the antimicrobial has antifungal properties. In some embodiments, the antimicrobial can be selected from Table 1.

TABLE 1
List of Antimicrobials

| Antimicrobial | |
|---|---|
| Class | Example(s) of Antimicrobial Agent |
| Aminocyclitols | Apramycin, Hygromycin B, Spectinomycin |
| Aminoglycosides | Amikacin, Arbekacin, Bekanamycin, Dibekacin, Dihydrostreptomycin, |
| | Framycetin, Gentamicin, Isepamicin, Kanamycin, Neomycin, |
| | Netilmicin, Paromomycin, Ribostamycin, Robostamycin, Sisomicin, |
| | Streptomycin, Tobramycin |
| Beta-lactamase | Avibactam, Clavulanic acid, Sulbactam, Tazobactam, Vaborbactam |
| inhibitors | |
| Carbapenems | Biapenem, Doripenem, Ertapenem, Faropenem, Imipenem, Meropenem, |
| | Panipenem |
| Cephalosporins- | Cefacetrile, Cefadroxil, Cefalonium, Cefaloridine, Cefazedone, |
| first generation | Cefazolin, Cefradine (Cephradine), Cefroxadine, Ceftezole, Cephalexin |
| | (Cefalexin), Cephalothin (Cefalotin), Cephapirin (Cefapirin, Cefapyrin) |
| Cephalosporins- | Cefaclor, Cefamandole, Cefatrizine, Cefbuperazone, Cefmetazole, |
| second generation | Cefminox, Cefonicide, Ceforanide, Cefotetan, Cefotiam, Cefoxitin, |
| | Cefprozil, Cefsulodin, Cefuroxime, Lorcarbef (Loracarbef, Lorcacarbef) |
| Cephalosporins- | Cefcapene, Cefdinir, Cefditoren, Cefetamet, Cefixime, Cefmenoxime, |
| third generation | Cefodizime, Cefoperazone, Cefoselis, Cefotaxime, Cefovecin, |
| | Cefpiramide, Cefpodoxime, Ceftazidime, Ceftibuten, Ceftiofur, |
| | Ceftizoxime, Ceftriaxone, Flomoxef, Latamoxef |
| Cephalosporins- | Cefepime, Cefozopran, Cefpirome, Cefquinome |
| fourth generation | |
| Cephalosporins- | Ceftaroline, Ceftobiprole, Ceftolozane |
| fifth generation | |
| Coumarins | Novobiocin |
| Diaminopyrimidines | Baquiloprim, Brodimoprim, Iclaprim, Ormetoprim, Tetroxoprim, |
| | Trimethoprim |
| Fluoroquinolones | Besifloxacin, Ciprofloxacin, Danofloxacin, Delafloxacin, Difloxacin, |
| | Enoxacin, Enrofloxacin, Finafloxacin, Gatifloxacin, Gemifloxacin, |
| | Grepafloxacin, Ibafloxacin, Levofloxacin, Lomefloxacin, |
| | Marbofloxacin, Moxifloxacin, Norfloxacin, Ofloxacin, Orbifloxacin, |
| | Pazufloxacin, Pefloxacin, Pradofloxacin, Prulifloxacin, Rufloxacin, |
| | Sitafloxacin, Sparfloxacin, Temafloxacin, Trovafloxacin |
| | (Alatrofloxacin) |
| Fosfomycins | Fosfomycin |
| Fusidic acid | Fusidic acid |
| Glycopeptides | Avoparcin, Dalbavancin, Oritavancin, Teicoplanin, Telavancin, |
| | Vancomycin |
| Glycylcylines | Tigecycline |
| Ketolides | Telithromycin |
| Lincosamides | Clindamycin, Lincomycin, Pirlimycin |
| Lipopeptides | Daptomycin |
| Macrocyclics | Fidaxomicin |
| Macrolides | Azithromycin, Carbomycin, Clarithromycin, Dirithromycin, |
| | Erythromycin, Flurithromycin, Gamithromycin, Josamycin, |
| | Kitasamycin, Midecamycin, Miocamycin, Mirosamycin, Oleandomycin, |
| | Rokitamycin, Roxithromycin, Solithromycin, Spiramycin, |
| | Terdecamycin, Tildipirosin, Tilmicosin, Troleandomycin, |
| | Tulathromycin, Tylosin, Tylvalosin |
| Monobactams | Aztreonam, Carumonam |
| Nitrofurans | Furaltadone, Furazolidone, Nifurtoinol, Nitrofural, Nitrofurantoin |
| Nitroimidazoles | Benznidazole, Metronidazole, Ornidazole, Secnidazole, Tinidazole |
| Orthosomycins | Avilamycin |
| Oxazolidinones | Linezolid, Tedizolid |
| Penicillins | Amoxicillin, Ampicillin, Aspoxicillin, Azidocillin, Azlocillin, |
| | Bacampicillin, Benzathine penicillin, Carbenicillin, Carindacillin, |
| | Clomethocillin (Nafcillin), Cloxacillin, Dicloxacillin, Epicillin, |
| | Flucloxacillin, Hetacillin, Mecillinam, Metampicillin, Methicillin, |
| | Mezlocillin, Oxacillin, Penamecillin, Penethamate hydriodine, Penicillin |
| | G (Benzylpenicillin), Penicillin V (Phenoxymethylpenicillin), |
| | Pheneticillin, Piperacillin, Pivampicillin, Pivmicillam, Propicillin, |
| | Sulbenicillin, Sultamicillin, Talampicillin, Temocillin, Ticarcillin, |
| | Tobicillin |
| Phenicols | Chloramphenicol, Florfenicol, Thiamphenicol |
| Pleuromutilins | Retapamulin, Tiamulin, Valnemulin |
| Polymyxins | Colistimethate, Colistin (Polymyxin E), Polymyxin B |
| Polypeptides | Bacitracin, Enramycin (Enduracidin), Gramicidin |
| Pseudomonic acids | Mupirocin |
| Quinolones | Cinoxacin, Fleroxacin, Flumequin, Garenoxacin, Miloxacin, Nalidixic |
| | acid, Oxolinic acid, Ozenoxacin, Pipemidic acid, Piromidic acid, |
| | Rosoxacin |
| Rifamycins | Rifabutin, Rifampin (Rifampicin), Rifamycin, Rifapentine, Rifaximin |
| Streptogramins | Dalfopristin, Pristinamycin, Quinupristin, Virginiamycin |
| Sulphonamides | Formosulfathiazole, Ormosulfathiazole, Phthalylsulfathiazole, |
| | Sulfacetamide, Sulfachloropyridazine, Sulfadiazine, Sulfadimerazin, |
| | Sulfadimethoxine, Sulfadimidine, Sulfadoxine, Sulfafurazole, |
| | Sulfaguanidine, Sulfaisodimidine, Sulfalene, Sulfamazone, |
| | Sulfamerazine, Sulfamethazine, Sulfamethizole, Sulfamethoxazole, |
| | Sulfamethoxine, Sulfamethoxypyridazine, Sulfametomidine, |
| | Sulfametoxydiazine, Sulfametrole, Sulfamonomethoxine, Sulfamoxole, |
| | Sulfanilamide, Sulfaperin, Sulfaphenazole, Sulfapyridine, |
| | Sulfaquinoxaline, Sulfasalazine, Sulfathiazole, Sulfathiourea, |
| | Sulfisoxazole, Sulfonamide |
| Tetracyclines | Chlortetracycline, Clomocycline, Demeclocycline, Doxycycline, |
| | Lymecycline, Metacycline, Minocycline, Oxytetracycline, |
| | Penimepicycline, Rolitetracycline, Tetracycline |
| Therapeutic agents | 4-Aminosalicylic Acid (Para-aminosalicylic acid), Bedaquiline, |
| for tuberculosis | Calcium aminosalicylate, Capreomycin, Cycloserine, Ethambutol, |
| | Ethionamide, Isoniazid, Morinamide, Protionamide, Pyrazinamide, |
| | Terizidone, Tiocarlide, Viomycin |
| Aminocoumarins | Novobiocin |
| Arsenical | Roxarsone, Nitarsone |
| Bicyclomycin | Bicozamycin |
| Orthosomycins | Avilamycin |
| Phosphoglycolipids | Bambermycin (=Flavomycin) |
| Ionophores | lasalocid, Monensin, Narasin, Salinomycin |
| (including | |
| polyesthers) | |
| Quinoxalines | Carbadox, Olaquindox |

### Assessment of Treatment of the Skin Injury

It can take up to 5-7 days for a skin flap or a graft to adhere to the wound bed of the skin injury. However, it can take only about 36-48 hours for epithelialization to begin at the injured edge of the skin injury. Once epithelialization has occurred, it is expected that the skin injury would stop leaking fluid. If the wound ceases draining through a non-occlusive device disclosed herein, the injury is likely healed or healing.

In some embodiments, the method comprises monitoring or observing a marker of healing without removing the device from the skin injury. In some embodiments, the marker of healing comprises a cessation of wound drainage from the skin injury, a cell layer of closure at an injured edge of the skin injury, an indicator of skin viability, or any combination thereof. In some embodiments, the indicator of skin viability comprises blanching of the skin injury with pressure, pink coloration of the skin injury, or any combination thereof.

### Subjects

In some embodiments, the subject is human. In some embodiments, the subject is a mammal. In some embodiments, the subject is a farm animal, domesticated animal, pig, horse, mule, donkey, cow, sheep, goat, alpaca, zoo animal, non-human primate, monkey, ape, gorilla, panda, big cat, lion, tiger, leopard, cheetah, fox, wolf, hyena, bear, elephant, giraffe, rhinoceros, hippo, zebra, deer, moose, companion animal, cat, or dog.

In some embodiments, the subject is at increased risk for traumatic skin injuries or skin tears. In some embodiments, the skin of the subject is at risk of tearing due to shear, friction, or blunt forces. In some embodiments, the subject is elderly, critically ill, critically injured, or malnourished. In some embodiments, the subject has fragile skin. In some embodiments, the skin of the subject is too fragile for staples, sutures or wound strips to successfully treat a skin injury.

### Methods of Making

Provided herein is a method of making a skin wound dressing, the method comprising applying an adhesive layer to a bottom side of a mesh scaffold and adding a plurality of fenestrations to the mesh scaffold or adhesive layer configured to allow for fluid egress when the skin wound dressing is placed over the skin injury of a subject. In some embodiments, the adhesive layer comprises an adhesive tape, film, glue, spray, gel, foam, or any combination thereof.

The fenestration pattern can be configured to preserve immobilization capacity of the skin wound dressing. In some cases, the method comprising at least one of punching, cutting, stamping, puncturing, burning, melting, or lasering a plurality of fenestrations through at least one of the mesh scaffold and adhesive layer. A fenestration of the plurality of fenestrations can have a linear shape, a rectangular shape, a circular shape, an elliptical shape, a triangular shape, a trapezoidal shape, or a cross shape. In some cases, the method comprises arranging the plurality of fenestrations in a regular pattern having an offset distance between a first fenestration and a second fenestration. In some cases, the method comprises arranging the plurality of fenestrations in an irregular pattern.

In some embodiments, the method further comprises affixing a backing layer to the adhesive layer. The backing layer can be configured to removably attach to the adhesive layer. The backing layer can help with storage of the skin wound dressing or application of the skin wound dressing to a skin injury.

In some embodiments, the method further comprises applying a non-adherent overlay to a top side of the mesh scaffold. The non-adherent overlay can prevent adhesion of the skin wound dressing to, for example, an absorbent covering (e.g., bandage, gauze, textile, sleeve, etc.) placed over the skin wound dressing to absorb the wound exudate. The absorbent covering can be changed as needed and without removing the skin wound dressing from the subject. In some cases, the non-adherent overlay reduces the adhesion of the absorbent covering to the skin wound dressing compared to a skin wound dressing with-out the non-adherent overlay.

### Kits

Described herein are kits for treating a skin injury of a subject, comprising: (a) a device comprising an adhesive material and a scaffold, wherein the scaffold is configured to structurally support a skin injury of a subject, and wherein the adhesive material is configured to affix the scaffold directly over the skin injury for at least 2 days; and (b) a protocol for use of the device. Also described herein are kits for treating a skin injury of a subject, comprising: (a) a device comprising a scaffold, wherein the scaffold is configured to structurally support a skin injury of a subject; (b) an adhesive material configured to affix the scaffold directly over the skin injury of the subject for at least 2 days; and (c) a protocol for use of the device and the adhesive material. In some embodiments, the kit or device further comprises a wound care product, and wherein the kit comprises a protocol for administration or application of the wound care product to the subject or the skin injury of the subject. In some embodiments, the wound care product comprises an antimicrobial, a silver antimicrobial, a silver material, an antimicrobial gel foam, an active ingredient, an antibiotic, an antifungal, a biologic, a medicament, an antiseptic, an anesthetic, an analgesic, a therapeutic agent, a hemostatic agent, or any combination thereof.

In some embodiments, the device is non-occlusive. In some embodiments, the kit further comprises a tool configured to add fenestrations to the device. In some embodiments, the tool is configured to cut, puncture, penetrate, pierce or stamp fenestrations through the device. The tool can be used to add fenestrations to a device of the kit.

In some embodiments, the device does not adhere or minimally adheres to a wound dressing when affixed directly over a skin injury of a subject. In some embodiments, the kit comprises a non-adherent overlay layer (e.g., a non-adherent overlay film or spray). The non-adherent overlay layer can be applied to a device of the kit before or after the device is affixed directly over the skin injury of the subject. The non-adherent overlay layer can be applied to the device to prevent the device from adhering to a covering (e.g., a dressing) while the device is affixed directly over the skin injury.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A non-occlusive device comprising:
a) a first layer comprising an adhesive material; and
b) a second layer comprising a scaffold configured to structurally support a skin injury of a subject,
wherein the first layer is configured to affix the second layer directly over the skin injury of the subject for at least 2 days.

2. A non-occlusive device comprising:
a) an adhesive material; and
b) a scaffold configured to structurally support a skin injury of a subject,
wherein the adhesive material is configured to affix the scaffold directly over the skin injury of the subject for at least 2 days.

3. The non-occlusive device of aspect 1 or 2, wherein the non-occlusive device is configured to provide primary closure or delayed primary closure of the skin injury of the subject.

4. The non-occlusive device of any one of aspects 1-3, wherein the non-occlusive device is a primary closure device.

5. The non-occlusive device of any one of aspects 1-4, wherein the non-occlusive device is self-adhering to the skin injury of the subject.

6. The non-occlusive device of any one of aspects 1-5, wherein the non-occlusive device provides multiplanar or multiaxial immobilization of the skin injury of the subject.

7. The non-occlusive device of any one of aspects 1-6, wherein the non-occlusive device prevents disruption of wound repair by immobilizing the skin injury of the subject.

8. The non-occlusive device of any one of aspects 1-7, wherein the non-occlusive device prevents disruption of wound repair due to shear forces, blistering, subdermal fluid accumulation, hematoma, sub graft fluid accumulation, or any combination thereof.

9. The non-occlusive device of any one of aspects 1-8, wherein at least one of the non-occlusive device, the first layer, the adhesive material, the second layer, or the scaffold is configured to cover, enclose, or envelop at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the skin injury or an area of skin proximate to, surrounding, and comprising the skin injury.

10. The non-occlusive device of any one of aspects 1-9, wherein the skin injury comprises a skin tear, a skin flap, an avulsed skin flap, a skin laceration, a skin blister, a skin graft, a tissue graft, a biocompatible material graft, a skin abrasion, a skin incision, or any combination thereof.

11. The non-occlusive device of any one of aspects 1-10, wherein the skin injury comprises an acute wound, a subacute wound, or a chronic wound.

12. The non-occlusive device of any one of aspects 1-11, wherein the skin injury comprises a separation or uncoupling of a first skin layer from a second skin layer.

13. The non-occlusive device of any one of aspects 1-12, wherein the skin injury comprises a separation or uncoupling of: (i) an epidermis of the subject from a dermis of the subject; (ii) the epidermis and the dermis from an underlying bodily tissue of the subject; or (iii) any combination thereof, wherein the underlying bodily tissue comprises at least one of fat, muscle, or fascia of the subject underlying or adjacent to the skin injury.

14. The non-occlusive device of any one of aspects 1-13, wherein the non-occlusive device, the first layer, the adhesive material, the second layer, the scaffold, a third layer, or any combination thereof is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% transparent.

15. The non-occlusive device of any one of aspects 1-14, wherein the first layer, the adhesive material, the second layer, the scaffold, a third layer, or any combination thereof comprises a transparent material.

16. The non-occlusive device of any one of aspects 1-15, wherein the non-occlusive device is configured to permit egress of fluid from the skin injury associated with wound repair without removing the non-occlusive device from the skin injury of the subject.

17. The non-occlusive device of any one of aspects 1-16, wherein the non-occlusive device is configured to be worn when the subject takes a shower or a bath.

18. The non-occlusive device of any one of aspects 1-17, wherein the first layer or the adhesive material is configured to affix the scaffold directly over the skin injury of the subject for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days.

19. The non-occlusive device of any one of aspects 1-18, wherein the non-occlusive device is configured to removably attach to the skin injury via the adhesive material.

20. The non-occlusive device of any one of aspects 1-19, wherein the adhesive material comprises a tape, a film, a glue, a spray, a gel, a foam, or any combination thereof.

21. The non-occlusive device of any one of aspects 1-20, wherein the adhesive material is biocompatible.

22. The non-occlusive device of any one of aspects 1-21, wherein the adhesive material is elastic.

23. The non-occlusive device of any one of aspects 1-22, wherein the non-occlusive device is configured to affix to the subject without providing staples or sutures to the skin injury, the non-occlusive device, the subject, or any combination thereof.

24. The non-occlusive device of any one of aspects 1-23, wherein the non-occlusive device further comprises a non-adhesive backing layer attached to the adhesive material.

25. The non-occlusive device of aspect 24, wherein the non-adhesive backing layer is configured to be removed from the non-occlusive device before affixing the second layer or the scaffold directly over the skin injury of the subject.

26. The non-occlusive device of any one of aspects 1-25, wherein the non-occlusive device comprises a plurality of fenestrations.

27. The non-occlusive device of any one of aspects 1-26, wherein the non-occlusive device comprises a plurality of fenestrations through the first layer, the adhesive material, the second layer, the scaffold, a third layer, or any combination thereof.

28. The non-occlusive device of aspect 26 or 27, wherein the plurality of fenestrations allows for egress of any fluid that develops from or adjacent to the skin injury, wherein the fluid is associated with wound injury or repair.

29. The non-occlusive device of any one of aspects 26-28, wherein the plurality of fenestrations is at least one of punched, cut, stamped, punctured, formed, sprayed, seared, burned, melted, or lasered through the first layer, the adhesive material, the second layer, the scaffold, a third layer, or any combination thereof.

30. The non-occlusive device of any one of aspects 1-29, wherein the non-occlusive device comprises a fenestration pattern.

31. The non-occlusive device of aspect 30, wherein the fenestration pattern is configured to preserve immobilization capacity or tensile strength of at least one of the first layer, the adhesive material, the second layer, the scaffold, or the non-occlusive device.

32. The non-occlusive device of aspect 30 or 31, wherein the fenestration pattern comprises at least one of a substantially linear, a substantially rectangular, a substantially circular, a substantially elliptical, a substantially triangular, or a substantially trapezoidal shape.

33. The non-occlusive device of any one of aspects 30-32, wherein the fenestration pattern comprises a grid of shapes.

34. The non-occlusive device of aspect 33, wherein the grid of shapes comprises a plurality of substantially linear shapes, a plurality of substantially rectangular shapes, a plurality of substantially circular shapes, a plurality of substantially elliptical shapes, a plurality of substantially triangular shapes, a plurality of substantially trapezoidal shapes, or any combination thereof.

35. The non-occlusive device of any one of aspects 1-34, wherein the scaffold provides tensile strength, durability, structure, elasticity, or any combination thereof to the non-occlusive device.

36. The non-occlusive device of any one of aspects 1-35, wherein the scaffold bolsters or provides multiplanar or multiaxial immobilization of the skin injury of the subject.

37. The non-occlusive device of any one of aspects 1-36, wherein the scaffold comprises a polymer, a nanofiber, a microfiber, a fiber, a metal, a wire, a string, a mesh, a fabric, a polyester, a textile, a foam, a gel foam, or any combination thereof.

38. The non-occlusive device of any one of the aspects 1-37, wherein the scaffold comprises a weave of polymers, nanofibers, microfibers, fibers, metals, wires, strings, meshes, fabrics, polyesters, textiles, foams, gel foams, or any combination thereof.

39. The non-occlusive device of any one of aspects 1-38, wherein the scaffold comprises a biocompatible material, a biologic, or any combination thereof.

40. The non-occlusive device of any one of aspects 1-39, wherein the scaffold comprises a biocompatible mesh.

41. The non-occlusive device of any one of aspects 1-40, wherein the scaffold comprises a synthetic material.

42. The non-occlusive device of any one of aspects 1-41, wherein the scaffold comprises an elastic material.

43. The non-occlusive device of any one of aspects 1-42, wherein the scaffold comprises a plurality of pores.

44. The non-occlusive device of aspect 43, wherein the plurality of pores comprises an average pore size between 0.2 mm and 20 mm.

45. The non-occlusive device of aspect 43 or 44, wherein the plurality of pores allows for egress of any fluid that develops from or adjacent to the skin injury, wherein the fluid is associated with wound injury or repair.

46. The non-occlusive device of any one of aspects 1-45, wherein the scaffold is configured to permit egress of fluid from the skin injury associated with wound repair without removing the scaffold from the skin injury of the subject.

47. The non-occlusive device of any one of aspects 1-46, wherein the non-occlusive device further comprises a third layer configured to prevent at least one of a bandage, a compression bandage, a dressing, an absorbent pad, an absorbent material, a sleeve, a compression sleeve, a covering, a drape, a fabric, a textile, a gown, or an article of clothing contacting the non-occlusive device from adhering or affixing to the non-occlusive device.

48. The non-occlusive device of aspect 47, wherein the third layer is coupled to at least one of the first layer, the adhesive material, the second layer, or the scaffold.

49. The non-occlusive device of aspect 47 or 48, wherein the third layer comprises a non-adhesive overlay, material, surface, or any combination thereof.

50. The non-occlusive device of aspect 49, wherein at least one of the third layer, the non-adhesive overlay, the non-adhesive material, or the non-adhesive surface comprises a tape, a film, a glue, a spray, a gel, a foam, or any combination thereof.

51. The non-occlusive device of any one of aspects 1-50, wherein the non-occlusive device is configured to be removed from the skin injury of the subject when the skin injury exhibits a marker of healing, the skin injury is attached to a wound bed of the subject, or any combination thereof.

52. The non-occlusive device of any one of aspects 1-51, wherein the non-occlusive device or scaffold is configured to be removed from the skin injury immediately or after at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 13 minutes, at least 14 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days from when the skin injury exhibits the marker of healing.

53. The non-occlusive device of aspect 51 or 52, wherein the marker of healing comprises a cessation of wound drainage from the skin injury, a cell layer of closure at an injured edge of the skin injury, an indicator of skin viability, blanching of the skin injury with pressure, pink coloration of the skin injury, or any combination thereof.

54. The non-occlusive device of any one of aspects 1-53, wherein the non-occlusive device comprises a surface that is configured to contact the subject without affixing to the subject for at least 2 days.

55. The non-occlusive device of any one of aspects 1-54, wherein at least one of the first layer, the adhesive material, the second layer, or the scaffold comprises a surface that is configured to contact the subject without affixing to the subject for at least 2 days.

56. The non-occlusive device of any one of aspects 1-55, wherein the first layer or the adhesive material is partially applied, minimally applied, absent, partially removed, or fully removed from a surface of the second layer or the scaffold, and wherein the surface of the second layer or the scaffold is configured to contact the subject.

57. The non-occlusive device of any one of aspects 1-56, wherein the first layer, the adhesive material, the second layer, the scaffold, the non-occlusive device, or any combination thereof, comprises at least one of an antimicrobial, a silver antimicrobial, a silver material, an antimicrobial gel foam, an active ingredient, an antibiotic, an antifungal, a wound care product, a biologic, a medicament, an antiseptic, an anesthetic, an analgesic, a therapeutic, a hemostatic agent, or any combination thereof.

58. The non-occlusive device of aspect 57, wherein the antimicrobial comprises at least one of an over-the-counter antimicrobial treatment, a prescribed antimicrobial treatment, silver, a silver antimicrobial, or a gel foam material with antimicrobial properties.

59. The non-occlusive device of aspect 57 or 58, wherein the antimicrobial is selected from Table 1.

60. The non-occlusive device of any one of aspects 1-59, wherein the subject is a mammal, human, non-human primate, farm animal, domesticated animal, pig, horse, mule, donkey, cow, sheep, goat, alpaca, zoo animal, monkey, ape, gorilla, panda, big cat, lion, tiger, leopard, cheetah, fox, wolf, hyena, bear, elephant, giraffe, rhinoceros, hippo, zebra, deer, moose, companion animal, cat, or dog.

61. A method for treating a skin injury of a subject, the method comprising:
a) affixing a non-occlusive device directly over the skin injury of the subject, wherein the non-occlusive device comprises:
   i. a first layer comprising an adhesive material; and
   ii. a second layer comprising a scaffold configured to structurally support the skin injury of the subject,
wherein the first layer is configured to affix the second layer directly over the skin injury of the subject for at least 2 days.

62. A method for treating a skin injury of a subject, the method comprising:
a) applying an adhesive material to the subject; and
b) affixing a non-occlusive device directly over the skin injury of the subject, wherein the non-occlusive device comprises a scaffold configured to structurally support the skin injury,
wherein the adhesive material is configured to affix the non-occlusive device directly over the skin injury of the subject for at least 2 days.

63. The method of aspect 61 or 62, wherein the non-occlusive device is configured to provide primary closure or delayed primary closure of the skin injury of the subject.

64. The method of any one of aspects 61-63, further comprising providing primary closure or delayed primary closure of the skin injury with the non-occlusive device.

65. The method of any one of aspects 61-64, wherein the non-occlusive device is self-adhering to the skin injury of the subject.

66. The method of any one of aspects 61-65, further comprising providing multiplanar or multiaxial immobilization of the skin injury of the subject with the non-occlusive device.

67. The method of any one of aspects 61-66, further comprising preventing disruption of wound repair by immobilizing the skin injury of the subject with the non-occlusive device.

68. The method of any one of aspects 61-67, further comprising preventing disruption of wound repair due to shear forces, blistering, subdermal fluid accumulation, hematoma, sub graft fluid accumulation, or any combination thereof with the non-occlusive device.

69. The method of any one of aspects 61-68, further comprising covering, enclosing, or enveloping at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the skin injury or an area of skin proximate to, surrounding, and comprising the skin injury with at least one of the non-occlusive device, the first layer, the adhesive material, the second layer, or the scaffold.

70. The method of any one of aspects 61-69, wherein the skin injury comprises a skin tear, a skin flap, an avulsed skin flap, a skin laceration, a skin blister, a skin graft, a tissue graft, a biocompatible material graft, a skin abrasion, a skin incision, or any combination thereof.

71. The method of any one of aspects 61-70, wherein the skin injury comprises an acute wound, a subacute wound, or a chronic wound.

72. The method of any one of aspects 61-71, further comprising applying a graft to the skin injury, the non-occlusive device, or any combination thereof before affixing the non-occlusive device directly over the skin injury of the subject.

73. The method of aspect 72, wherein the graft comprises a biocompatible graft, a skin graft, a tissue graft, a composite graft, a cell graft, a biocompatible material graft, a synthetic skin graft, a synthetic tissue graft, an exoskeletal support piece, or any combination thereof.

74. The method of aspect 72 or 73, wherein the graft comprises a split-thickness graft, a full-thickness graft, a composite graft, or any combination thereof.

75. The method of any one of aspects 72-74, further comprising providing multiplanar or multiaxial immobilization to the graft with the non-occlusive device or the scaffold.

76. The method of any one of aspects 72-75, further comprising preventing shear forces, blistering, subdermal fluid accumulation, hematoma, sub graft fluid accumulation, or any combination thereof from disrupting attachment of the graft to a wound bed of the subject with the non-occlusive device or the scaffold.

77. The method of any one of aspects 61-76, further comprising monitoring or observing a marker of healing without removing the non-occlusive device from the skin injury.

78. The method of any one of aspects 61-77, further comprising monitoring or observing direct repair of the skin injury without removing the non-occlusive device from the skin injury.

79. The method of any one of aspects 61-78, wherein the non-occlusive device, the first layer, the adhesive material, the second layer, the scaffold, a third layer, or any combination thereof is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% transparent.

80. The method of any one of aspects 61-79, wherein the adhesive material, the scaffold, a third layer, or any combination thereof comprises a transparent material.

81. The method of any one of aspects 61-80, further comprising covering the non-occlusive device with an absorbent dressing, wherein the dressing comprises at least one of a bandage, gauze, pad, absorbent pad, sleeve, compression sleeve, compression bandage, wound covering, fabric, or textile.

82. The method of aspect 81, further comprising changing the absorbent dressing without removing, changing, or displacing the non-occlusive device.

83. The method of aspect 81 or 82, further comprising permitting fluid from the skin injury of the subject associated with wound repair to pass through the non-occlusive device to the dressing.

84. The method of any one of aspects 61-83, further comprising affixing the second layer or the scaffold directly over the skin injury of the subject continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days.

85. The method of any one of aspects 61-84, further comprising affixing the scaffold directly over the skin injury of the subject using an adhesive material comprising a tape, a film, a glue, a spray, a gel, a foam, or any combination thereof.

86. The method of any one of aspects 61-85, further comprising providing staples or sutures to at least one of the skin injury, a skin layer, the subcutis, the dermis, or the epidermis of the subject before affixing the non-occlusive device directly over the skin injury of the subject.

87. The method of any one of aspects 61-86, further comprising performing deep tissue closure with sutures before affixing the non-occlusive device directly over the skin injury of the subject.

88. The method of any one of aspects 61-87, further comprising providing staples or sutures to the non-occlusive device, the subject, the skin injury, a skin layer of the subject, or any combination thereof after affixing the non-occlusive device directly over the skin injury of the subject.

89. The method of any one of aspects 61-88, further comprising removing a non-adhesive backing layer attached to the adhesive material before affixing the non-occlusive device directly over the skin injury of the subject.

90. The method of any one of aspects 61-89, wherein the non-occlusive device comprises a plurality of fenestrations through at least one of the adhesive material, the scaffold, or a third layer.

91. The method of aspect 90, wherein the plurality of fenestrations allows for egress of any fluid that develops from or adjacent to the skin injury, wherein the fluid is associated with wound injury or repair.

92. The method of aspect 90 or 91, wherein the plurality of fenestrations is at least one of punched, cut, stamped, punctured, formed, sprayed, seared, burned, melted, or lasered through the first layer, the adhesive material, the second layer, the scaffold, a third layer, or any combination thereof.

93. The method of any one of aspects 61-92, wherein the non-occlusive device comprises a fenestration pattern.

94. The method of any one of aspects 61-93, further comprising adding a plurality of fenestrations or a fenestration pattern to the adhesive material, the first layer, the scaffold, the second layer, a third layer, or any combination thereof.

95. The method of aspect 93 or 94, wherein the fenestration pattern is configured to preserve immobilization capacity or tensile strength of at least one of the first layer, the adhesive material, the second layer, the scaffold, or the non-occlusive device.

96. The method of any one of aspects 93-95, wherein the fenestration pattern comprises at least one of a substantially linear, a substantially rectangular, a substantially circular, a substantially elliptical, a substantially triangular, or a substantially trapezoidal shape.

97. The method of any one of aspects 93-96, wherein the fenestration pattern comprises a grid of shapes.

98. The method of aspect 97, wherein the grid of shapes comprises a plurality of substantially linear shapes, a plurality of substantially rectangular shapes, a plurality of substantially circular shapes, a plurality of substantially elliptical shapes, a plurality of substantially triangular shapes, a plurality of substantially trapezoidal shapes, or any combination thereof.

99. The method of any one of aspects 61-98, wherein the scaffold provides tensile strength, durability, structure, elasticity, or any combination thereof to the non-occlusive device.

100. The method of any one of aspects 61-99, wherein the scaffold bolsters or provides multiplanar or multiaxial immobilization of the skin injury of the subject.

101. The method of any one of aspects 61-100, wherein the scaffold comprises a polymer, a nanofiber, a microfiber, a fiber, a metal, a wire, a string, a mesh, a fabric, a polyester, a textile, a foam, a gel foam, or any combination thereof.

102. The method of any one of aspects 61-101, wherein the scaffold comprises a biocompatible material, a biologic, or any combination thereof.

103. The method of any one of aspects 61-102, wherein the scaffold comprises a biocompatible mesh.

104. The method of any one of aspects 61-103, wherein the scaffold comprises a plurality of pores.

105. The method of aspect 104, wherein the plurality of pores comprises an average pore size between 0.2 mm and 20 mm.

106. The method of aspect 104 or 105, wherein the plurality of pores allows for egress of any fluid that develops from or adjacent to the skin injury, wherein the fluid is associated with wound injury or repair.

107. The method of any one of aspects 61-106, wherein the scaffold is configured to permit egress of fluid from the skin injury associated with wound repair without removing the scaffold from the skin injury of the subject.

108. The method of any one of aspects 61-107, further comprising preventing at least one of a bandage, a compression bandage, a dressing, an absorbent pad, an absorbent material, a sleeve, a compression sleeve, a covering, a drape, a fabric, a textile, a gown, or an article of clothing contacting the non-occlusive device from adhering to the non-occlusive device.

109. The method of aspect 108, wherein the third layer comprises a non-adhesive overlay, a non-adhesive material, a non-adhesive surface, or any combination thereof.

110. The method of any one of aspects 61-109, further comprising removing the non-occlusive device from the skin injury of the subject when the skin injury exhibits a marker of healing, the skin injury is attached to a wound bed of the subject, or any combination thereof.

111. The method of any one of aspects 61-110, further comprising removing the non-occlusive device or scaffold from the skin injury immediately or after at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 13 minutes, at least 14 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 31 days from when the skin injury exhibits the marker of healing.

112. The method of aspect 110 or 111, wherein the marker of healing comprises a cessation of wound drainage from the skin injury, a cell layer of closure at an injured edge of the skin injury, an indicator of skin viability, blanching of the skin injury with pressure, pink coloration of the skin injury, or any combination thereof.

113. The method of any one of aspects 61-112, further comprising removing the non-occlusive device from the skin injury of the subject via a free edge of the non-occlusive device.

114. The method of any one of aspects 61-113, further comprising removing the non-occlusive device in a directionality relative to the skin injury that prevents reinjury of the skin injury.

115. The method of any one of aspects 61-114, further comprising orienting or positioning a free edge of the non-occlusive device relative to the skin injury before affixing the non-occlusive device directly over the skin injury of the subject.

116. The method of aspect 115, wherein the free edge is configured to contact the subject without affixing to the subject for at least 2 days.

117. The method of any one of aspects 61-116, further comprising providing an antimicrobial, a silver antimicrobial, a silver material, an antimicrobial gel foam, an active ingredient, an antibiotic, an antifungal, a wound care product, a biologic, a medicament, an antiseptic, an anesthetic, an analgesic, a therapeutic agent, a hemostatic agent, or any combination thereof to or adjacent to the skin injury of the subject.

118. The method of aspect 117, wherein the antimicrobial comprises at least one of an over-the-counter antimicrobial treatment, a prescribed antimicrobial treatment, silver, a silver antimicrobial, or a gel foam material with antimicrobial properties.

119. The method of aspect 117 or 118, wherein the antimicrobial is selected from Table 1.

120. The method of aspect 119, further comprising applying the antimicrobial to the skin injury before affixing the non-occlusive device directly over the skin injury of the subject.

121. The method of any one of aspects 61-120, further comprising cleaning the skin injury and removing non-viable debris from the skin injury before affixing the non-occlusive device directly over the skin injury of the subject.

122. The method of any one of aspects 61-121, further comprising positioning a wound edge of the skin injury to approximate anatomical position before affixing the non-occlusive device directly over the skin injury of the subject.

123. The method of any one of aspects 61-122, wherein the subject is a mammal, human, non-human primate, farm animal, domesticated animal, pig, horse, mule, donkey, cow, sheep, goat, alpaca, zoo animal, monkey, ape, gorilla, panda, big cat, lion, tiger, leopard, cheetah, fox, wolf, hyena, bear, elephant, giraffe, rhinoceros, hippo, zebra, deer, moose, companion animal, cat, or dog.

124. A kit comprising:
a. a device comprising an adhesive material and a scaffold, wherein the scaffold is configured to structurally support a skin injury of a subject, and wherein the adhesive material is configured to affix the scaffold directly over the skin injury for at least 2 days; and
b. a protocol for use of the device.

125. A kit comprising:
a. a device comprising a scaffold, wherein the scaffold is configured to structurally support a skin injury of a subject;
b. an adhesive material configured to affix the scaffold directly over the skin injury of the subject for at least 2 days; and
c. a protocol for use of the device and the adhesive material.

126. The kit of aspect 124 or 125, wherein the kit or the device comprises a wound care product, and wherein the kit comprises a protocol for administration or application of the wound care product to the subject or the skin injury of the subject.

127. The kit of aspect 126, wherein the wound care product comprises an antimicrobial, a silver antimicrobial, a silver material, an antimicrobial gel foam, an active ingredient, an antibiotic, an antifungal, a biologic, a medicament, an antiseptic, an anesthetic, an analgesic, a therapeutic agent, a hemostatic agent, or any combination thereof.

128. The kit of any one of aspects 124-127, wherein the device is non-occlusive.

129. The kit of any one of aspects 124-128, wherein the kit further comprises a tool configured to add fenestrations to the device.

### EXAMPLES

### Example 1: Treatment of a Large Skin Tear on the Elbow of a Subject

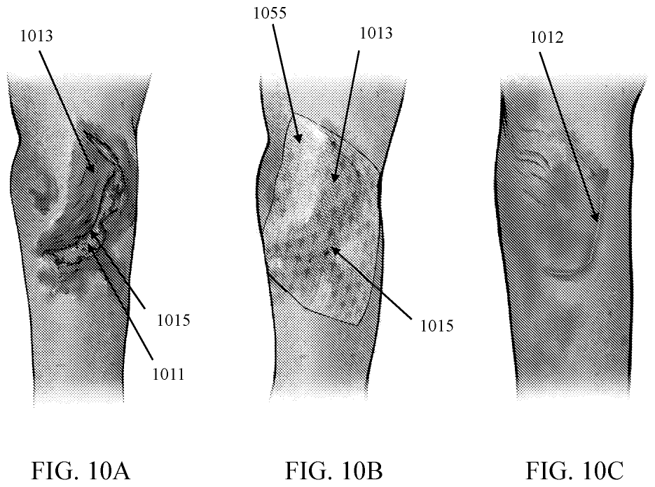

FIGS. 10A-10C depict a large skin injury treated using a device and a method described herein. In some embodiments, the skin injury comprises a large skin tear 1011 comprising a skin flap 1013, wherein the skin flap comprises an injured edge 1015, shown in FIG. 10A. The devices provided herein are configured to be adaptable to complex wound shape and topography. In some embodiments, after initial cleaning, debridement, and treatment with antimicrobials of the skin tear 1011, a non-occlusive device 1055 of the present disclosure can be applied directly over the skin injury of the subject, using the non-occlusive device 1055 to pull and hold the injured edge 1015 of the skin flap 1013 into alignment with normal anatomical position, as shown in FIG. 10B. After 2 weeks, the non-occlusive device can be removed and the healed skin injury 1012 will be closed and no longer draining fluid, as shown in FIG. 10C.

### Example 2: Treatment of Minor Skin Trauma in a Subject

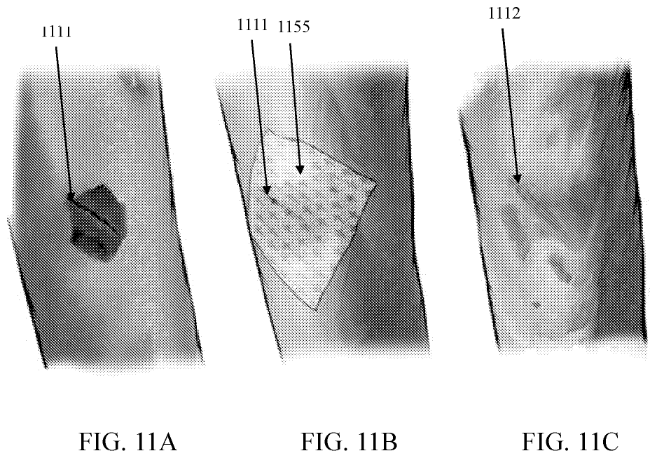

FIGS. 11A-11C depict treatment of a minor skin injury of a subject using a device and a method described herein. FIG. 11A depicts an exemplary skin laceration 1111 before treatment. A non-occlusive device 1155 of the present disclosure can be affixed directly over the skin laceration as shown in FIG. 11B, and the non-occlusive device can be left in place for a total of about 7 days. After 2 days of covering the non-occlusive device with a bandage, the subject could no longer need to cover the device 1155 with dressing and the subject can allow the wound and non-occlusive device to get wet in the shower without adversely affecting use of the non-occlusive device. The non-occlusive device can be removed after 7 days, and the healed skin laceration 1112 will be closed, as depicted in FIG. 11C.

### Example 3: Treatment of a Total Degloving of a Forearm of a Subject

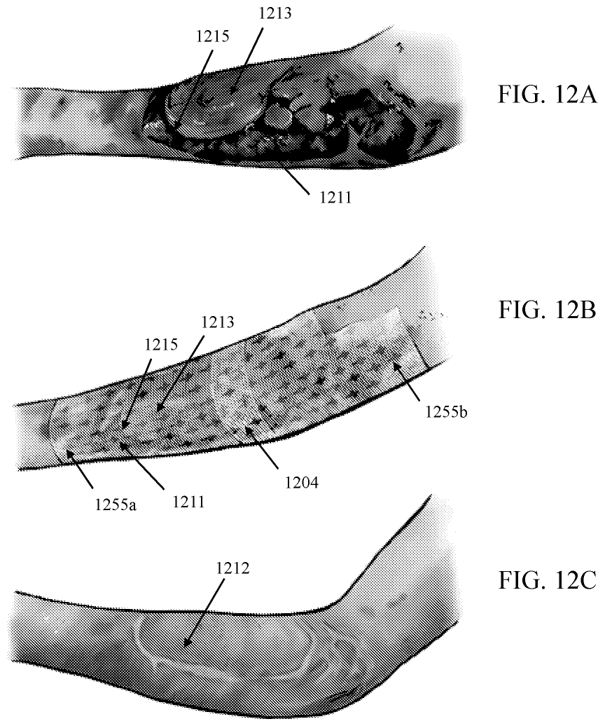

FIGS. 12A-12C depict treatment of a total degloving of a forearm of a subject using a device and a method provided herein. A severe skin tear 1211 comprising a partial skin flap 1213 is depicted as a full thickness wound, shown in FIG. 12A. The partial skin flap 1213 comprises an injured edge 1215. Two sheets of a non-occlusive device 1255a and 1255b as provided herein can be affixed directly over the skin tear 1211 while bringing the partial skin flap 1213 as close to anatomical position as possible, shown in FIG. 12B. The two sheets of the non-occlusive device 1255a and 1255b can overlap in area 1204. After 2 weeks, the non-occlusive device can be removed from the healed skin injury 1212 and all the tissue of the partial skin flap can still be viable, seen in FIG. 12C. Visible markers of healing can include pink coloration of the skin, a cell layer of closure at the injured edge 1215 of the partial skin flap 1213, or a cessation of wound draining. The non-occlusive device will successfully close the wound and allow for complete survival of the injured skin after 14 days.

### Example 4: Single Step Application of the Device to Skin Injuries

A non-occlusive device comprising an adhesive material and a scaffold as described herein was used to treat eleven skin injuries of ten subjects. The skin injuries were classified as skin tears either having no tissue loss (e.g., a full skin flap where the edges of the skin flap can be realigned to the normal anatomical position without undue stretching) or partial tissue loss (e.g., a partial skin flap where the edges cannot be realigned to the normal anatomical position). For each skin injury treated, the non-occlusive device was affixed directly over the skin flap for at least seven days without removal. Gauze bandaging could be used during treatment to cover the non-occlusive device and such bandaging could be removed and replaced as necessary. Patient characteristics and treatment results are summarized in Table 2 below.

TABLE 2
Characteristics of patients treated with a non-occlusive device

| Injury | | Area | Treatment | % |
|---|---|---|---|---|
| ID | | Treated | Duration | Tissue |
| Number | Injury Classification | (centimeters) | (Days) | Survival |
| 001 | Skin tear without | 2 × 3 | 7 | 100 |
| | tissue loss | | | |
| 002 | Skin tear with partial | 8 × 4 | 8 | 100 |
| | tissue loss | | | |
| 003 | Skin tear with partial | 7 × 3 | 7 | 100 |
| | tissue loss | | | |
| 004 | Skin tear with partial | 11 × 11 | 7 | 100 |
| | tissue loss | | | |
| 005 | Skin tear with partial | 31 × 15 | 7 | 100 |
| | tissue loss | | | |
| 006 | Skin tear without | 16 × 2 | 7 | 100 |
| | tissue loss | | | |
| 007 | Skin tear with partial | 4 × 3 | 7 | 100 |
| | tissue loss | | | |
| 008 | Skin tear with partial | 5 × 3 | 7 | 100 |
| | tissue loss | | | |
| 009a | Skin tear without | 2 × 2 | 7 | 100 |
| | tissue loss | | | |
| 009b | Skin tear with partial | 2 × 3 | 7 | 100 |
| | tissue loss | | | |
| 010 | Skin tear without | 10 × 1 | 7 | 100 |
| | tissue loss | | | |

Upon return to the clinic to remove the non-occlusive device, each skin injury was evaluated to assess the survival of the skin flap. Results: Complete tissue survival was observed for each skin flap treated with a non-occlusive device and method as described herein.

Before the non-occlusive device was removed, the degree of edge lift (i.e., the degree to which the surface area adjacent to the device's edges that had detached from the subject during the treatment period) was measured and scored on a scale from 1 to 5, where 1 represents a minimal degree of edge lift and 5 represents a large degree of edge lift. During removal, the ease with which the non-occlusive device could be removed from the skin injury was measured and scored on a scale from 1 to 5, where 1 represents an easy removal and 5 represents a difficult removal (e.g., causing injury). Results: Each non-occlusive device received a score of 1 for degree of edge lift; and each non-occlusive device received a score of 1 for ease of removal.

### Example 5: Treatment of a Skin Tear Comprising a Full Skin Flap

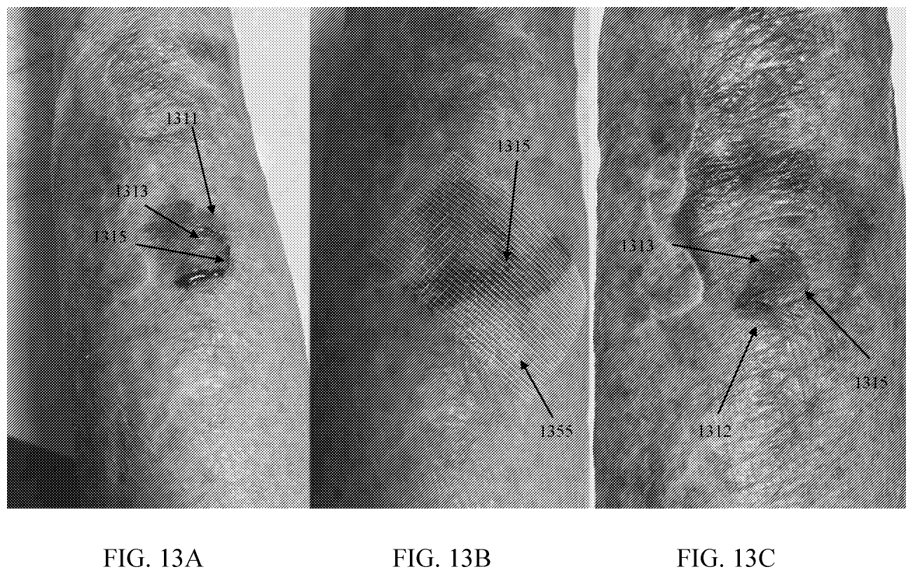

FIGS. 13A-13C depict a skin injury fully treated using a device and a method described herein. The subject presented to the clinic with a minor skin tear 1311 comprising a full skin flap 1313, shown in FIG. 13A. The skin flap comprises an injured edge 1315. The wound was first washed and clean. Then, the injured edge 1315 was realigned to normal anatomical position and a piece of a non-occlusive device 1355 of the present disclosure was affixed directly over the skin injury, as shown in FIG. 13B. After seven days, the non-occlusive device was removed from the healed skin injury 1312 without reinjuring the skin injury, as shown in FIG. 13C. Following removal of the non-occlusive device, the skin flap tissue 1313 sustained attachment to the underlying wound bed. A cell layer of closure could be observed at the injured edge 1315. Other markers of wound healing were also observed (e.g., a pink coloration could be seen surrounding the skin flap) and the skin flap tissue 1313 was deemed to be completely viable. The healed skin injury 1312 was closed and no longer draining fluid.

### Example 6: Treatment of a Hematoma and Skin Tear of a Subject

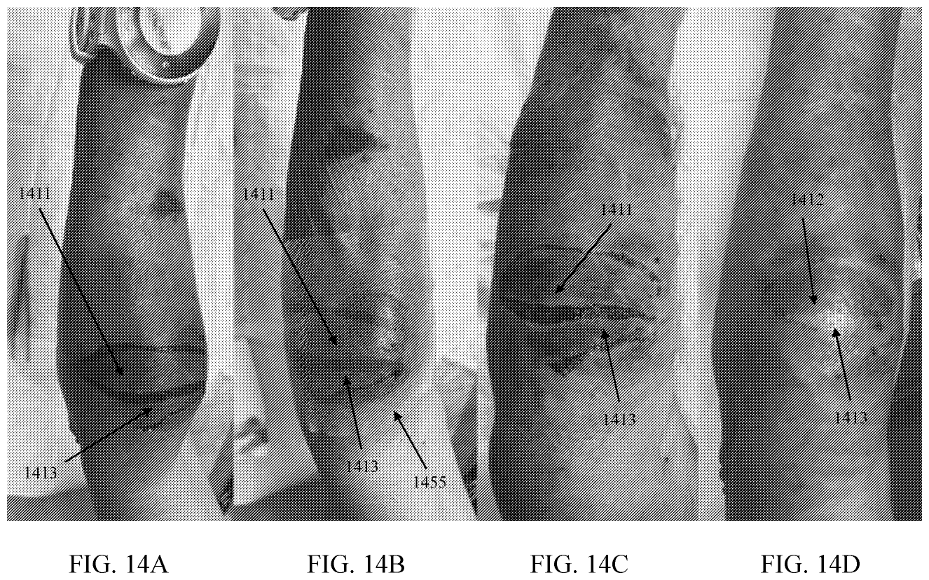

FIGS. 14A-14D depict treatment of a skin tear comprising a partial skin flap using a non-occlusive device and a method provided herein. The subject presented to the clinic with a skin tear 1411 having a partial skin flap 1413 and an underlying hematoma, shown in FIG. 14A. The skin tear was cleaned and a sheet of a non-occlusive device 1455 as provided herein was affixed directly over the skin tear 1411, shown in FIG. 14B. The non-occlusive device is configured to prevent shear forces and forces of the underlying hematoma from disrupting wound repair while bringing the partial skin flap 1413 as close to anatomical position as possible. After seven days of treatment, the non-occlusive device was removed from the skin injury 1411 and all the skin tissue of the partial skin flap 1413 was found to be viable, shown in FIG. 14C. Visible markers of healing (e.g., spots of pink coloration) could be observed after one week. Following removal of the non-occlusive device, a light bandage or covering was applied over the skin injury 1411 to keep the tissue clean as it continued to heal. After three weeks, the treated skin injury 1412 had ceased draining and all the skin flap tissue 1413 remained viable, shown in FIG. 14D.

### Example 7: Treatment of a Skin Injury Comprising Two Partial Skin Flaps

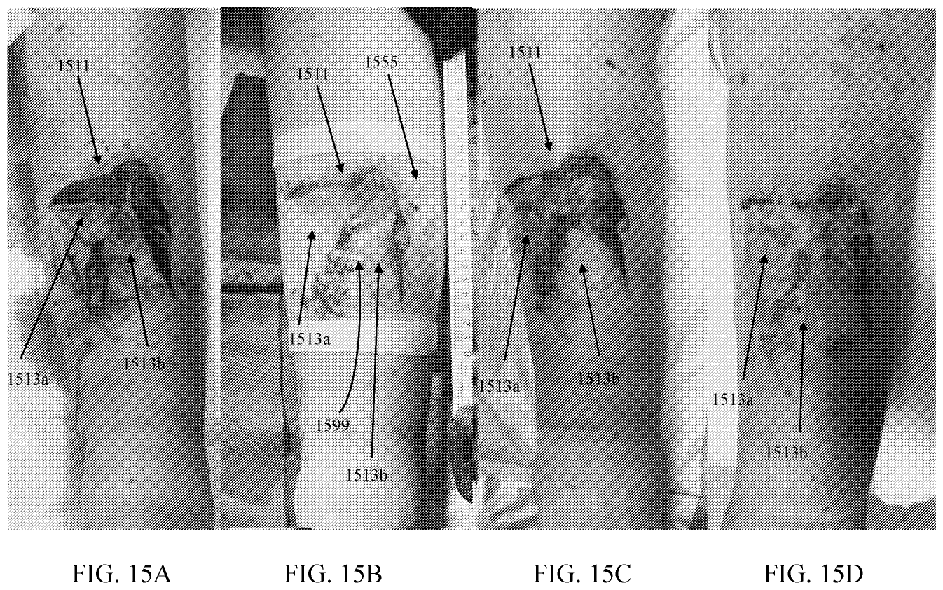

FIGS. 15A-15D depict treatment of a skin injury comprising two partial skin flaps using a non-occlusive device and a method provided herein. The subject presented to the clinic with a traumatic avulsion skin injury of the pretibial surface 1511 comprising a first partial skin flap 1513a and a second partial skin flap 1513b, shown in FIG. 15A. After the injury was washed and cleaned, several sutures 1599 were used to position the first and second skin flaps to come together, and then a sheet of a non-occlusive device 1555 as provided herein was affixed directly over the skin injury 1511, shown in FIG. 15B. The skin tissue of the first and second skin flaps is fragile and could easily rip at or near the sutures 1599 if a force (e.g., a shear force) is applied to pull one apart from the other. The non-occlusive device is configured to prevent shear forces from disrupting wound repair and can also prevent the skin flaps from being pulled apart while sutured together. After seven days of treatment, the non-occlusive device and sutures were removed and all the skin tissue of both skin flaps 1513a and 1513b was found to be viable, shown in FIG. 15C. Following removal of the non-occlusive device and sutures, a light bandage or covering was applied over the skin injury 1511 as it continued healing. After three weeks, all the tissue of the first and second skin flaps remained viable, shown in FIG. 15D.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

What is claimed:

1. A skin wound dressing comprising
a mesh scaffold having a top side and a bottom side, and
an adhesive layer associated with the bottom side of the scaffold,
wherein at least one of the mesh scaffold and adhesive layer comprise a plurality of fenestrations configured to allow for fluid egress when the dressing is placed over a skin injury of a subject.

2. The skin wound dressing of claim 1, wherein the mesh scaffold comprises a non-absorbable mesh.

3. The skin wound dressing of claim 1 or 2, wherein the mesh scaffold comprises an absorbable mesh.

4. The skin wound dressing of any one of claims 1 to 3, wherein the mesh scaffold comprises at least one of a polyester-based, polypropylene-based, silk-based, nylon-based, spandex-based, polyglactin-based and polyglycolic acid (PGA)-based mesh.

5. A skin wound dressing comprising
a polyester-based or polypropylene-based mesh scaffold having a top side and a bottom side, and
an adhesive layer associated with the bottom side of the scaffold,
wherein the dressing has a porosity configured to allow for fluid egress when the dressing is placed over a skin injury of a subject.

6. The skin wound dressing of claim 5, wherein at least one of the mesh scaffold and the adhesive layer comprises a plurality of fenestrations.

7. The skin wound dressing of any one of preceding claims, wherein the mesh scaffold has a low absorbance or no absorbance of wound exudate from the skin injury.

8. The skin wound dressing of claim 7, wherein the mesh scaffold absorbs less than 50% of wound exudate from the skin injury.

9. The skin wound dressing of any one of preceding claims, wherein the mesh scaffold has a low biodegradability when the dressing is placed over the skin injury.

10. The skin wound dressing of claim 9, wherein the mesh scaffold maintains at least 50% of one or more mechanical properties after 1, 2, 3, 4, 5, 6, or 7 days of covering the skin injury as compared to the mesh scaffold before covering the skin injury.

11. The skin wound dressing of claim 10, wherein the one or more mechanical properties comprises tensile modulus, tensile strength, tensile strain, elastic modulus, stiffness, or any combination thereof.

12. The skin wound dressing of any one of preceding claims, wherein the porosity is about 0.001 mm to about 20 mm, about 0.01 mm to about 10 mm, about 0.05 mm to about 5 mm, or about 0.1 mm to about 5 mm.

13. The skin wound dressing of any one of claim 1-4 or 6-12, wherein a fenestration of the plurality of fenestrations has a linear shape, a rectangular shape, a circular shape, an elliptical shape, a triangular shape, a trapezoidal shape, or a cross shape.

14. The skin wound dressing of any one of claim 1-4 or 6-13, wherein the plurality of fenestrations is arranged in a regular pattern having an offset distance between a first fenestration and a second fenestration.

15. The skin wound dressing of any one of claim 1-4 or 6-14, wherein the plurality of fenestrations is arranged in an irregular pattern.

16. The skin wound dressing of any one of claim 1-4 or 6-15, wherein the plurality of fenestrations is configured to allow for fluid egress when the dressing is placed over the skin injury.

17. The skin wound dressing of any one of the preceding claims, wherein the adhesive layer is configured to adhere the device to the subject for at least 1, 2, 3, 4, 5, 6, or 7 days.

18. The skin wound dressing of any one of the preceding claims, wherein the skin injury comprises a skin flap or a skin graft.

19. The skin wound dressing of claim 18, wherein the dressing provides multiplanar or multiaxial immobilization of the skin flap or the skin graft to the skin injury.

20. The skin wound dressing of any one of preceding claims, wherein a surface area of the skin injury is at least 1, 2, 3, 4, 5, 6, 7, 8, 9, 10, 15, or 20 cm².

21. The skin wound dressing of claim 18 or 19, wherein a surface area of the skin flap or the skin graft is at least 1, 2, 3, 4, 5, 6, 7, 8, 9, 10, 15, or 20 cm².

22. The skin wound dressing of claim 20 or 21, wherein the dressing is configured to adhere across the surface area of the skin injury continuously for at least 1, 2, 3, 4, 5, 6, or 7 days.

23. The skin wound dressing of any one of the preceding claims, wherein the adhesive layer comprises an adhesive tape, film, glue, spray, gel, foam, or any combination thereof applied to the bottom side of the mesh scaffold.

24. The skin wound dressing of any one of preceding claims, wherein the mesh scaffold has a pore size between about 0.01 mm to about 10 mm, about 0.05 mm to about 5.0 mm, about 0.8 mm to about 4.0 mm, or about 1.0 mm to about 2.0 mm.

25. The skin wound dressing of any one of the preceding claims, wherein the mesh scaffold has a weight of between about 5 grams per square meter (GSM) to about 200 GSM.

26. The skin wound dressing of any one of the preceding claims, wherein the mesh scaffold comprises a thickness of between about 0.01 mm to about 10 mm, about 0.05 mm to about 5 mm, or about 0.1 mm to about 1 mm.

27. The skin wound dressing of any one of claim 1-4 or 6-26, wherein the plurality of fenestrations is at least one of punched, cut, stamped, punctured, formed, sprayed, seared, burned, melted, and lasered through the mesh scaffold, the adhesive layer, a third layer (e.g., a non-adherent overlay layer), or any combination thereof.

28. The skin wound dressing of any one of claim 1-4 or 6-27, wherein the fenestration pattern is configured to preserve immobilization capacity of the dressing.

29. The skin wound dressing of any one of the preceding claims, further comprising a non-adhesive backing layer attached to the adhesive layer.

30. The skin wound dressing of any one of the preceding claims, further comprising a non-adherent overlay layer attached to the top side of the scaffold and configured to prevent at least one of a bandage, a compression bandage, an absorbent covering, a drape, a textile, and an article of clothing contacting the skin wound dressing from at least partially adhering to the dressing.

31. The skin wound dressing of any one of the preceding claims, wherein the dressing comprises an antimicrobial, a silver antimicrobial, a silver material, an antimicrobial gel foam, an active ingredient, an antibiotic, an antifungal, a wound care product, a biologic, a medicament, an antiseptic, an anesthetic, an analgesic, a therapeutic, a hemostatic agent, or any combination thereof.

32. The skin wound dressing of any one of the preceding claims, wherein the mesh scaffold is woven or knitted.

33. A method of treating a skin injury comprising a wound bed, the method comprising:
a. covering a skin flap of the skin injury or a skin graft applied to the wound bed of the skin injury with a piece of a skin wound dressing, wherein the skin wound dressing comprises:
i. a mesh scaffold having a top side and a bottom side; and
ii. an adhesive layer associated with the bottom side of the scaffold,
wherein the skin wound dressing has a porosity configured to allow for fluid egress when the skin wound dressing is placed over a skin injury of a subject; and
b. providing multiplanar or multiaxial immobilization of the skin flap or the skin graft with the skin wound dressing.

34. The method of claim 33, wherein at least one of the mesh scaffold and the adhesive layer of the skin wound dressing comprises a plurality of fenestrations.

35. A method of treating a skin injury of a subject comprising:
a. adhering a piece of a skin wound dressing directly over the skin injury, the skin wound dressing comprising:
i. a mesh scaffold having a top side and a bottom side; and
ii. an adhesive layer associated with the bottom side of the scaffold,
wherein the at least one of the mesh scaffold and adhesive layer comprises a plurality of fenestrations configured to allow for fluid egress when the skin wound dressing is placed over the skin injury of the subject; and
b. providing multiplanar or multiaxial immobilization of the skin injury with the skin wound dressing.

36. The method of claim 35, wherein the skin injury comprises a skin flap or a skin graft atop a wound bed of the skin injury.

37. The method of any one of claims 33-36, wherein the mesh scaffold comprises a non-absorbable mesh.

38. The method of any one of claims 33-37, wherein the mesh scaffold comprises an absorbable mesh.

39. The method of any one of claims 33-38, wherein the mesh scaffold comprises at least one of a polyester-based, polypropylene-based, silk-based, nylon-based, spandex-based, polyglactin-based and polyglycolic acid (PGA)-based mesh.

40. The method of any one of claims 33-39, wherein the mesh scaffold absorbs less than 50% of wound exudate from the skin injury.

41. The method of any one of claims 33-40, wherein the mesh scaffold has a low biodegradability when the skin wound dressing is placed over the skin injury.

42. The method of any one of claims 33-41, wherein the mesh scaffold maintains at least 50% of one or more mechanical properties after 1, 2, 3, 4, 5, 6, or 7 days of covering the skin injury as compared to the mesh scaffold before covering the skin injury.

43. The method of claim 42, wherein the one or more mechanical properties comprises tensile modulus, tensile strength, tensile strain, elastic modulus, stiffness, or any combination thereof.

44. The method of any one of claims 33-43, comprising providing multiplanar or multiaxial immobilization of the skin flap or the skin graft with the skin wound dressing until the skin flap or the skin graft is at least partially attached to the wound bed.

45. The method of any one of claims 33-44, comprising preventing disruption of wound repair due to shear forces, blistering, subdermal fluid accumulation, hematoma, sub graft fluid accumulation, or any combination thereof with the skin wound dressing.

46. The method of any one of claim 33-34 or 36-45, comprising covering at least about 50% to about 100% of the surface area of the skin flap or the skin graft with the piece of the skin wound dressing.

47. The method of any one of claims 33-46, comprising adhering the skin wound dressing across the skin injury continuously for at least 1, 2, 3, 4, 5, 6, or 7 days.

48. The method of any one of claim 33-34 or 36-47, comprising removing the skin wound dressing from the skin injury once the skin flap or skin graft is at least partially attached to the wound bed of the subject.

49. The method of any one of claims 33-48, comprising removing the skin wound dressing from the skin injury once the skin injury exhibits a marker of healing.

50. The method of claim 49, wherein the marker of healing comprises a cessation of wound drainage from the skin injury, a cell layer of closure at an injured edge of the skin injury, an indicator of skin viability, blanching of the skin injury with pressure, pink coloration of the skin injury, or any combination thereof.

51. A method of making a skin wound dressing, the method comprising applying an adhesive layer to a bottom side of a polyester-based or polypropylene-based mesh scaffold, wherein the skin wound dressing has a porosity configured to allow for fluid egress when the skin wound dressing is placed over the skin injury of a subject.

52. The method of claim 49, further comprising adding a plurality of fenestrations to the mesh scaffold or adhesive layer.

53. A method of making a skin wound dressing, the method comprising applying an adhesive layer to a bottom side of a mesh scaffold and adding a plurality of fenestrations to the mesh scaffold or adhesive layer configured to allow for fluid egress when the skin wound dressing is placed over the skin injury of a subject.

54. The method of claim 52 or 53, wherein the fenestration pattern is configured to preserve immobilization capacity of the skin wound dressing.

55. The method of any one of claims 51-54, wherein the adhesive layer comprises an adhesive tape, film, glue, spray, gel, foam, or any combination thereof.

56. The method of any one of claims 51-55, comprising affixing a backing layer to the adhesive layer.

57. The method of any one of claims 51-56, comprising applying a non-adhesive overlay to a top side of the mesh scaffold.
